(19) United States
(12) Patent Application Publication
(10) Pub. No.: US 2024/0094634 A1
(43) Pub. Date: Mar. 21, 2024

(54) RADIATION-SENSITIVE RESIN COMPOSITION AND METHOD OF FORMING RESIST PATTERN

(71) Applicant: JSR CORPORATION, Tokyo (JP)

(72) Inventors: Katsuaki NISHIKORI, Tokyo (JP); Takahiro KAWAI, Tokyo (JP)

(73) Assignee: JSR CORPORATION, Tokyo (JP)

(21) Appl. No.: 18/235,924

(22) Filed: Aug. 21, 2023

(30) Foreign Application Priority Data
Aug. 24, 2022 (JP) 2022-133175
Jun. 28, 2023 (JP) 2023-106331

(51) Int. Cl. G03F 7/039; G03F 7/004

(52) U.S. Cl. CPC G03F 7/039; G03F 7/0045

(57) ABSTRACT

A radiation-sensitive resin composition contains: a first polymer having a first structural unit including a partial structure obtained by substituting a hydrogen atom of a carboxy group or of a phenolic hydroxy group with an acid-labile group represented by formula (1); and a compound including: a monovalent radiation-sensitive onium cation moiety including an aromatic ring structure which includes a fluorine atom or a fluorine atom-containing group; and a monovalent organic acid anion moiety. Ar¹ represents a group obtained by removing one hydrogen atom from a substituted or unsubstituted aromatic ring structure having 5 to 30 ring atoms; R ¹ and R² each independently represent a substituted or unsubstituted monovalent aliphatic hydrocarbon group having 1 to 10 carbon atoms; and * denotes a site bonding to an ethereal oxygen atom in the carboxy group or an oxygen atom in the phenolic hydroxy group.

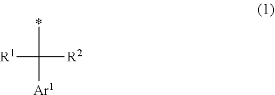

### CROSS-REFERENCE TO RELATED APPLICATIONS

The present application claims priority to Japanese Patent Application No. 2022-133175 filed Aug. 24, 2022, and to Japanese Patent Application No. 2023-106331 filed Jun. 28, 2023. The contents of these applications are incorporated herein by reference in their entirety.

### BACKGROUND OF THE INVENTION

### Field of the Invention

The present invention relates to a radiation-sensitive resin composition and a method of forming a resist pattern.

### Discussion of the Background

A radiation-sensitive resin composition for use in microfabrication by lithography generates an acid at light-exposed regions upon an irradiation with a radioactive ray, e.g.: an electromagnetic wave such as a far ultraviolet ray such as an ArF excimer laser beam (wavelength of 193 nm) or a KrF excimer laser beam (wavelength of 248 nm), or an extreme ultraviolet ray (EUV) (wavelength of 13.5 nm); or a charged particle ray such as an electron beam. A chemical reaction in which the acid serves as a catalyst causes a difference between the light-exposed regions and light-unexposed regions in rates of dissolution in a developer solution, whereby a resist pattern is formed on a substrate.

Such radiation-sensitive resin compositions are required not only to have favorable sensitivity to exposure light such as the extreme ultraviolet ray and the electron beam, but also to result in superiority in terms of CDU (Critical Dimension Uniformity) performance and resolution.

To meet these requirements, types, molecular structures, and the like of polymers, acid generating agents, and other components which may be used in radiation-sensitive resin compositions have been investigated, and combinations thereof have been further investigated in detail (see Japanese Unexamined Patent Applications, Publication Nos. 2010-134279, 2014-224984, and 2016-047815).

### SUMMARY OF THE INVENTION

According to an aspect of the present invention, a radiation-sensitive resin composition contains: a first polymer having a first structural unit including a partial structure obtained by substituting a hydrogen atom of a carboxy group or of a phenolic hydroxy group with an acid-labile group represented by formula (1); and a compound including: a monovalent radiation-sensitive onium cation moiety including an aromatic ring structure which includes a fluorine atom or a fluorine atom-containing group; and a monovalent organic acid anion moiety. Ar¹ represents a group obtained by removing one hydrogen atom from a substituted or unsubstituted aromatic ring structure having 5 to 30 ring atoms; R¹ and R² each independently represent a substituted or unsubstituted monovalent aliphatic hydrocarbon group having 1 to 10 carbon atoms; and * denotes a site bonding to an ethereal oxygen atom in the carboxy group or an oxygen atom in the phenolic hydroxy group.

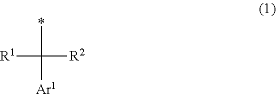

According to another aspect of the present invention, a method of forming a resist pattern includes: applying the above-described radiation-sensitive resin composition directly or indirectly on a substrate to form a resist film; exposing the resist film; and developing the resist film exposed.

### DESCRIPTION OF THE EMBODIMENTS

As used herein, the words “a” and “an” and the like carry the meaning of “one or more.” When an amount, concentration, or other value or parameter is given as a range, and/or its description includes a list of upper and lower values, this is to be understood as specifically disclosing all integers and fractions within the given range, and all ranges formed from any pair of any upper and lower values, regardless of whether subranges are separately disclosed. Where a range of numerical values is recited herein, unless otherwise stated, the range is intended to include the endpoints thereof, as well as all integers and fractions within the range. As an example, a stated range of 1-10 fully describes and includes the independent subrange 3.4-7.2 as does the following list of values: 1, 4, 6, 10.

According to one embodiment of the invention, a radiation-sensitive resin composition contains: a polymer (hereinafter, may be also referred to as “(A) polymer” or “polymer (A)”) having a structural unit including a partial structure obtained by substituting a hydrogen atom of a carboxy group or of a phenolic hydroxy group with an acid-labile group represented by the following formula (1); and a compound (hereinafter, may be also referred to as “(Z) compound” or “compound (Z)”) having: a monovalent radiation-sensitive onium cation moiety including an aromatic ring structure obtained by substituting at least one hydrogen atom with a fluorine atom or a fluorine atom-containing group; and a monovalent organic acid anion moiety.

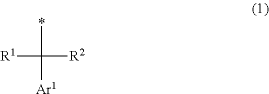

In the formula (1),
Ar¹ represents a group obtained by removing one hydrogen atom from a substituted or unsubstituted aromatic ring structure having 5 to 30 ring atoms;
R¹ and R² each independently represent a substituted or unsubstituted monovalent aliphatic hydrocarbon group having 1 to 10 carbon atoms; and
* denotes a site bonding to an ethereal oxygen atom in the carboxy group or an oxygen atom in the phenolic hydroxy group.

According to an other embodiment of the invention, a method of forming a resist pattern includes: applying the radiation-sensitive resin composition of the one embodiment of the present invention directly or indirectly on a substrate; exposing a resist film formed by the applying; and developing the resist film exposed.

The radiation-sensitive resin composition of the one embodiment of the present invention is superior in sensitivity, and results in superiority in CDU performance and resolution. The method of forming a resist pattern of the other embodiment of the present invention enables forming a resist pattern being superior in CDU performance and resolution with high sensitivity. Therefore, these can be suitably used in manufacturing processes of semiconductor devices, in which further progress of miniaturization is expected in the future.

Hereinafter, the radiation-sensitive resin composition and the method of forming a resist pattern of the embodiments of the present invention will be described in detail.

### Radiation-Sensitive Resin Composition

The radiation-sensitive resin composition contains the polymer (A) and the compound (Z). The radiation-sensitive resin composition typically contains an organic solvent (hereinafter, may be also referred to as “(D) organic solvent” or “organic solvent (D)”). The radiation-sensitive resin composition may contain, as a favorable component, a radiation-sensitive acid generating agent (hereinafter, may be also referred to as “(B) acid generating agent” or “acid generating acid (B)”) other than the compound (Z). The radiation-sensitive resin composition may contain, as a favorable component, an acid diffusion control agent (hereinafter, may be also referred to as “(C) acid diffusion control agent” or “acid diffusion control agent (C)”) other than the compound (Z). The radiation-sensitive resin composition may contain, as a favorable component, a polymer (hereinafter, may be also referred to as “(F) polymer” or “polymer (F)”) having a percentage content of fluorine atoms which is greater than that of the polymer (A). The radiation-sensitive resin composition may contain, within a range not leading to impairment of the effects of the present invention, other optional component(s).

Due to the polymer (A) and the compound (Z) being contained, the radiation-sensitive resin composition is superior in sensitivity, and results in superiority in CDU performance and resolution. Although not necessarily clarified and without wishing to be bound by any theory, the reason for achieving the aforementioned effects by the radiation-sensitive resin composition due to involving such a constitution may be presumed, for example, as in the following. Due to the polymer (A), which has a specific structural unit, described later, and the compound (Z), which has a specific cation structure, described later, being used in combination with each other, acid generation efficiency in light-exposed regions improves. It is considered that as a result, the radiation-sensitive resin composition is superior in sensitivity, and results in superiority in CDU performance and resolution.

The radiation-sensitive resin composition may be prepared, for example, by mixing the polymer (A) and the compound (Z), as well as the acid generating agent (B), the acid diffusion control agent (C), the organic solvent (D), the polymer (E), and/or the other optional component(s), which is/are added as needed, in a certain ratio, and preferably filtering a thus resulting mixture through a membrane filter having a pore size of no greater than 0.2 μm.

Each component contained in the radiation-sensitive resin composition is described below.

### (A) Polymer

The polymer (A) has a structural unit (hereinafter, may be also referred to as “structural unit (I)”) that includes an acid-labile group (hereinafter, may be also referred to as “acid-labile group (a)”) obtained by substituting a hydrogen atom of a carboxy group or of a phenolic hydroxy group with an acid-labile group represented by the formula (1), described later. The polymer (A) is a polymer, solubility of which in a developer solution is capable of being altered by an action of an acid. Due to the polymer (A) having the structural unit (I), the property of altering the solubility in a developer solution by an action of an acid is exhibited. The radiation-sensitive resin composition may contain one, or two or more types of the polymer (A).

It is preferred that the polymer (A) further has a structural unit (hereinafter, may be also referred to as “structural unit (II)”) that includes a phenolic hydroxy group. The polymer (A) may further have a structural unit (hereinafter, may be also referred to as “structural unit (III)”) that includes an acid-labile group other than the acid-labile group (a). The polymer (A) may further have other structural unit(s) (hereinafter, may be also referred to merely as “other structural unit(s)”), aside from the structural units (I) to (III). The polymer (A) can have one type, or two or more types of each of the structural units.

The lower limit of a proportion of the polymer (A) in the radiation-sensitive resin composition with respect to total components other than the organic solvent (D) contained in the radiation-sensitive resin composition is preferably 50% by mass, more preferably 70% by mass, and still more preferably 80% by mass. The upper limit of the proportion is preferably 99% by mass, and more preferably 95% by mass.

The lower limit of a polystyrene-equivalent weight average molecular weight (Mw) of the polymer (A) as determined by gel permeation chromatography (GPC) is preferably 1,000, more preferably 2,000, and still more preferably 3,000. The upper limit of the Mw is preferably 30,000, more preferably 20,000, and still more preferably 10,000. When the Mw of the polymer (A) falls within the above range, coating characteristics of the radiation-sensitive resin composition may be improved. The Mw of the polymer (A) can be adjusted by, for example, regulating the type, the amount, and the like of a polymerization initiator used in synthesis of the polymer (A).

The upper limit of a ratio (hereinafter may be also referred to as “Mw/Mn” or “polydispersity index”) of the Mw to a polystyrene-equivalent number average molecular weight (Mn) of the polymer (A) as determined by GPC is preferably 2.5, more preferably 2.0, and still more preferably 1.7. The lower limit of the ratio is typically 1.0, preferably 1.1, more preferably 1.2, and still more preferably 1.3.

### Method for Measuring Mw and Mn

As referred to herein, the Mw and Mn of the polymer are values measured by using gel permeation chromatography (GPC) under the following conditions.
GPC columns: “G2000 HXL”×2, “G3000 HXL”×1, and “G4000 HXL”×1, available from Tosoh Corporation
column temperature: 40 ° C.
elution solvent: tetrahydrofuran
flow rate: 1.0 mL/min
sample concentration: 1.0% by mass
amount of injected sample: 100 uL
detector: differential refractometer
standard substance: mono-dispersed polystyrene

The polymer (A) can be synthesized by, for example, polymerizing a monomer that gives each structural unit in accordance with a well-known procedure.

Each structural unit contained in the polymer (A) is described below.

### Structural Unit (I)

The structural unit (I) is a structural unit including a partial structure obtained by substituting a hydrogen atom of a carboxy group or of a phenolic hydroxy group with an acid-labile group (the acid-labile group (a)) represented by the following formula (1).

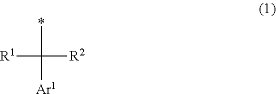

In the above formula (1),
Ar¹ represents a group obtained by removing one hydrogen atom from a substituted or unsubstituted aromatic ring structure having 5 to 30 ring atoms;
R¹ and R² each independently represent a substituted or unsubstituted monovalent aliphatic hydrocarbon group having 1 to 10 carbon atoms; and
* denotes a site bonding to an ethereal oxygen atom in the carboxy group or an oxygen atom in the phenolic hydroxy group.

The polymer (A) may have one, or two or more types of the structural unit (I).

The structural unit (I) is a structural unit that includes the acid-labile group (a). The “acid-labile group” as referred to herein means a group that substitutes for a hydrogen atom in a carboxy group, a hydroxy group, or the like, and is capable of being dissociated by an action of an acid to give a carboxy group, a hydroxy group, or the like. The acid-labile group (a) is a group obtained by substituting for a hydrogen atom contained in the carboxy group or the phenolic hydroxy group in the structural unit (I). In other words, the acid-labile group (a) in the structural unit (I) is bonded to the ethereal oxygen atom in the carbonyloxy group or the oxygen atom in the phenolic hydroxy group. The “phenolic hydroxy group” as referred to herein means a hydroxy group directly bonding to an aromatic ring in general, without being limited to a hydroxy group directly bonding to a benzene ring.

Due to the polymer (A) having the structural unit (I), the acid-labile group (a) is disassociated from the structural unit (I) by an action of the acid generated from the compound (Z), the acid generating agent (B), and/or the like upon exposure, whereby a difference is generated in the solubility of the polymer (A) in the developer solution, between light-exposed regions and light-unexposed regions, and thus forming a resist pattern is enabled. It is considered that the polymer (A) including the acid-labile group (a) in the structural unit (I) is one factor in the radiation-sensitive resin composition exhibiting superior sensitivity and resulting in superior CDU performance and resolution. Although not necessarily clarified and without wishing to be bound by any theory, it is presumed that the reason for this factor is that the acid-labile group (a) is easily dissociated by the action of the acid generated from the compound (Z), the acid generating agent (B), and/or the like upon the exposure, whereby the difference in the solubility of the polymer (A) in the developer solution between the light-exposed regions and the light-unexposed regions becomes greater.

The number of “ring atoms” as referred to herein means the number of atoms constituting a ring structure, and in the case of a polycyclic ring, the number of “ring atoms” means the number of atoms constituting the polycyclic ring. The “polycyclic ring” encompasses: a spiro-type polycyclic ring in which two rings have one shared atom; a fused polycyclic ring in which two rings have two shared atoms; and a ring-assembled polycyclic ring in which two rings are connected by a single bond without having any shared atom. The “ring structure” encompasses an “alicyclic structure” and an “aromatic ring structure”. The “alicyclic structure” encompasses an “aliphatic hydrocarbon ring structure” and an “aliphatic heterocyclic structure”. Of the alicyclic structures, polycyclic rings, including the aliphatic hydrocarbon ring structure and the aliphatic heterocyclic structure, fall under the “aliphatic heterocyclic structure”. The “aromatic ring structure” encompasses both an “aromatic hydrocarbon ring structure” and an “aromatic heterocyclic structure”. Of the aromatic structures, polycyclic rings, including the aromatic hydrocarbon ring structure and the aromatic heterocyclic structure, fall under the “aromatic heterocyclic structure”. A “group obtained by removing X hydrogen atoms from a ring structure” means a group obtained by removing X hydrogen atoms which bond to an atom which constitutes the ring structure.

The number of “carbon atoms” means the number of carbon atoms constituting a group. The valency means the number of atoms to which that group bonds. The “hydrocarbon group” encompasses an “aliphatic hydrocarbon group” and an “aromatic hydrocarbon group”. The “aliphatic hydrocarbon group” may be either a saturated hydrocarbon group or an unsaturated hydrocarbon group. From a different viewpoint, the “aliphatic hydrocarbon group” encompasses a “chain hydrocarbon group” and an “alicyclic hydrocarbon group”. The “chain hydrocarbon group” as referred to herein means a hydrocarbon group not having a ring structure but being constituted with only a chain structure, and may be exemplified by both a linear hydrocarbon group and a branched hydrocarbon group. The “alicyclic hydrocarbon group” as referred to herein means a hydrocarbon group having, as a ring structure, not an aromatic ring structure but an alicyclic structure alone, and may be exemplified by both a monocyclic alicyclic hydrocarbon group and a polycyclic alicyclic hydrocarbon group. With regard to this, it is not necessary for the alicyclic hydrocarbon group to be constituted with only an alicyclic structure; it may have a chain structure in a part thereof. The “aromatic hydrocarbon group” as referred to herein means a hydrocarbon group that includes an aromatic ring structure as a ring structure. With regard to this, it is not necessary for the aromatic hydrocarbon group to be constituted with only an aromatic ring, and it may have a chain structure or an alicyclic structure in a part thereof.

Examples of the aromatic ring structure having 5 to 30 ring atoms that gives Ar¹ include: an aromatic hydrocarbon ring structure having 6 to 30 ring atoms; an aromatic heterocyclic structure having 5 to 30 ring atoms; and the like.

Examples of the aromatic hydrocarbon ring structure having 6 to 30 ring atoms include: a benzene structure; fused polycyclic aromatic hydrocarbon ring structures such as a naphthalene structure, an anthracene structure, a fluorene structure, a biphenylene structure, and a phenanthrene structure; ring-assembled aromatic hydrocarbon rings such as a biphenyl structure, a terphenyl structure, a binaphthalene structure, and a phenylnaphthalene structure; and the like.

Examples of the aromatic heterocyclic structure having 5 to 30 ring atoms include: oxygen atom-containing heterocyclic structures such as a furan structure, a pyran structure, a benzofuran structure, and a benzopyran structure; nitrogen atom-containing heterocyclic structures such as a pyrrole structure, a pyridine structure, a pyrimidine structure, an indole structure, and a quinolone structure; sulfur atom-containing heterocyclic structures such as a thiophene structure and a dibenzothiophene structure; and the like.

The aromatic ring structure having 5 to 30 ring atoms that gives Ar¹ is preferably the aromatic hydrocarbon ring structure having 6 to 30 ring atoms, more preferably a benzene structure or the fused polycyclic aromatic hydrocarbon ring structure; and still more preferably a benzene structure or a naphthalene structure.

A part or all of hydrogen atoms bonded to atoms constituting the ring structure may be substituted with a substituent. Examples of the substituent include: halogen atoms such as a fluorine atom and an iodine atom; a hydroxy group; a carboxy group; a cyano group; a nitro group; an alkyl group, described later; a fluoroalkyl group (a group obtained by substituting a part or all of hydrogen atoms contained in the alkyl group with a fluorine atom); an alkoxy group; an alkoxycarbonyl group; an alkoxycarbonyloxy group; an acyl group; an acyloxy group; an oxo group (═O); and the like. Of these, the halogen atom, the alkyl group, the fluoroalkyl group, or the alkoxy group is preferred, and a fluorine atom, an iodine atom, a methyl group, a trifluoromethyl group, or a methoxy group is more preferred. In the case of the iodine atom, the sensitivity of the radiation-sensitive resin composition may be further improved.

Examples of the monovalent aliphatic hydrocarbon group having 1 to 10 carbon atoms which gives R¹ or R² include: a monovalent chain hydrocarbon group having 1 to 10 carbon atoms; a monovalent alicyclic hydrocarbon group having 3 to 20 carbon atoms; and the like.

Examples of the monovalent chain hydrocarbon group having 1 to 10 carbon atoms include: alkyl groups such as a methyl group, an ethyl group, an n-propyl group, an i-propyl group, an n-butyl group, a sec-butyl group, an isobutyl group, and a tert-butyl group; alkenyl groups such as an ethenyl group, a propenyl group, a butenyl group, and a 2-methylprop-1-en-1-yl group; alkynyl groups such as an ethynyl group, a propynyl group, and a butynyl group; and the like.

Examples of the monovalent alicyclic hydrocarbon group having 3 to 10 carbon atoms include: monocyclic alicyclic saturated hydrocarbon groups such as a cyclopropyl group, a cyclobutyl group, a cyclopentyl group, and a cyclohexyl group; polycyclic alicyclic saturated hydrocarbon groups such as a norbornyl group and an adamantyl group; monocyclic alicyclic unsaturated hydrocarbon groups such as a cyclopentenyl group and a cyclohexenyl group; polcyclic alicyclic unsaturated hydrocarbon groups such as a norbornenyl group; and the like.

The aliphatic hydrocarbon group that gives R¹ or R² is preferably the monovalent chain hydrocarbon group having 1 to 10 carbon atoms or the monovalent alicyclic hydrocarbon group having 3 to 20 carbon atoms, more preferably the alkyl group or the monocyclic alicyclic saturated hydrocarbon group, and still more preferably a methyl group, an ethyl group, an i-propyl group, or a cyclopropyl group.

A part or all of hydrogen atoms in the aliphatic hydrocarbon group may be substituted with a substituent. Examples of the substituent include groups similar to those exemplified as the substituent which may be contained in the ring structure which gives Ar¹, and the like. The substituent is preferably an alkoxy group.

The acid-labile group (a) is preferably a group obtained by substituting a hydrogen atom contained in the carboxy group in the structural unit (I). In other words, the acid-labile group (a) in the structural unit (I) preferably bonds to the ethereal oxygen atom in the carbonyloxy group.

The acid-labile group (a) is preferably a group represented by the following formulae (a-1) to (a-12).

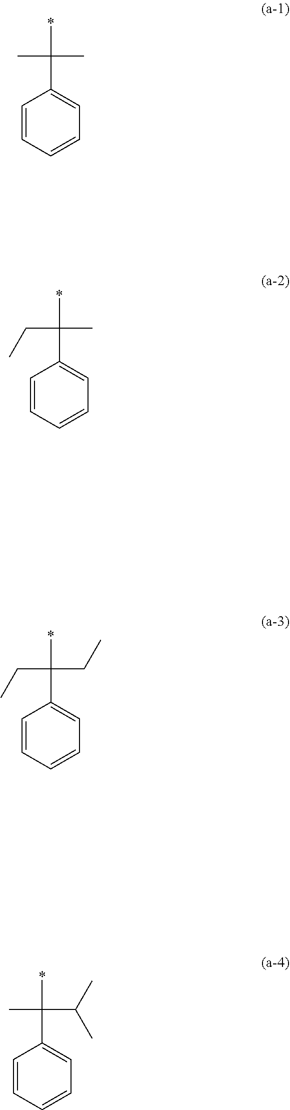

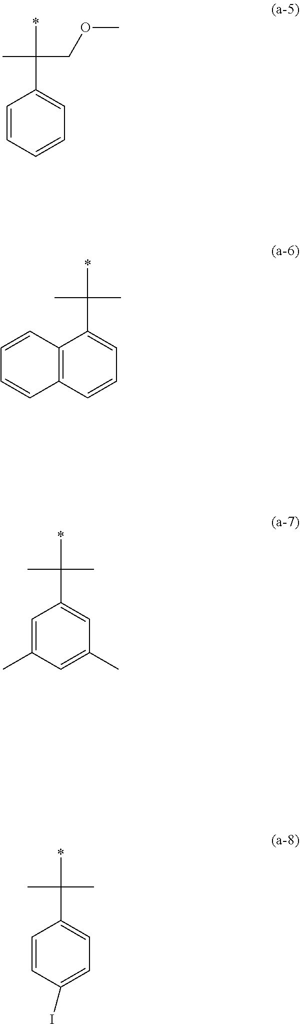

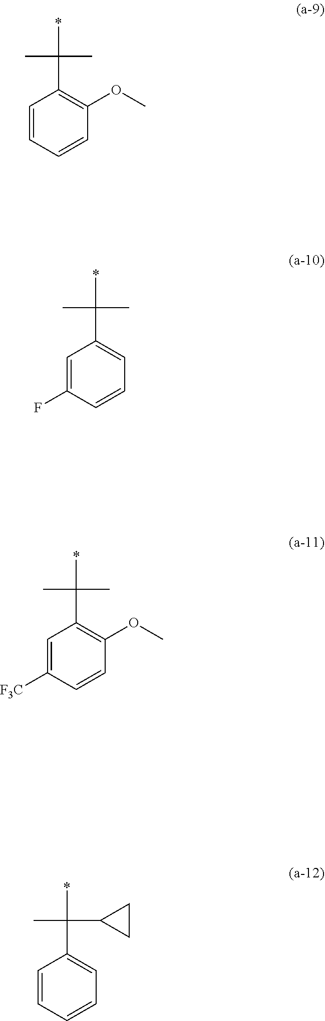

In the above formulae (a-1) to (a-12), * is as defined in the above formula (1).

Examples of the structural unit (I) include a structural unit (hereinafter, may be also referred to as “structural unit (I-1) or (I-2)”) represented by the following formula (3-1) or (3-2), and the like.

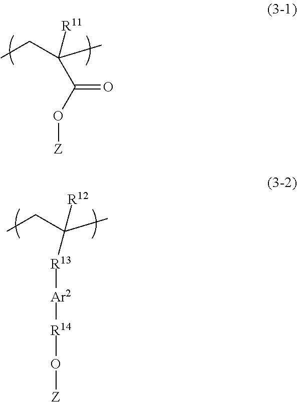

In the above formulae (3-1) and (3-2), Z represents the acid-labile group (acid-labile group (a)) represented by the above formula (1).

In the above formula (3-1), R¹¹ represents a hydrogen atom, a fluorine atom, a methyl group, or a trifluoromethyl group.

In the above formula (3-2), R¹² represents a hydrogen atom or a methyl group; R D represents a single bond, an oxygen atom, —COO—, or —CONH—; Ar² represents a group obtained by removing two hydrogen atoms from a substituted or unsubstituted aromatic hydrocarbon ring structure having 6 to 30 ring atoms; and R¹⁴ represents a single bond or —CO—.

In light of copolymerizability of a monomer that gives the structural unit (I), R¹¹ represents preferably a hydrogen atom or a methyl group, and more preferably a hydrogen atom.

R¹³ represents preferably a single bond or —COO—.

Examples of the aromatic hydrocarbon ring structure having 6 to 30 ring atoms which gives Ar² include, of the aromatic ring structures having 5 to 30 ring atoms which gives Ar¹ in the above formula (1), those similar to the ring structures exemplified as the aromatic hydrocarbon ring structure having 6 to 30 ring atoms, and the like. Of these, a benzene structure or a naphthalene structure is preferred.

R¹⁴ represents preferably —CO—.

The lower limit of a proportion of the structural unit (I) contained in the polymer (A) with respect to total structural units constituting the polymer (A) is preferably 1 mol %, more preferably 5 mol %, and still more preferably 10 mol %. The upper limit of the proportion is preferably 80 mol %, more preferably 70 mol %, and still more preferably 60 mol %. When the proportion of the structural unit (I) falls within the above range, the sensitivity of and the CDU performance and resolution resulting from the radiation-sensitive resin composition can be further improved. Furthermore, the lower limit may be preferably 20 mol %, may be more preferably 30 mol %, and may be still more preferably 40 mol %, and in these cases, the sensitivity and/or the resolution resulting from the radiation-sensitive resin composition may be further improved. It is to be noted that with respect to descriptions of the upper limit and the lower limit of numerical ranges as referred to herein, unless otherwise specified particularly, the upper limit may have the meaning of either “no greater than” or “less than”, and the lower limit may have the meaning of either “no less than” or “greater than”. Further, the upper limit value and the lower limit value may be combined ad libitum.

The polymer (A) having the structural unit (I) can be synthesized by polymerizing a monomer (hereinafter, may be also referred to as “(X) monomer” or “monomer (X)”) that gives the structural unit (I) by a well-known procedure.

### Structural Unit (II)

The structural unit (II) is a structural unit that includes a phenolic hydroxy group. The polymer (A) may include one, or two or more types of the structural unit (II).

In a case of conducting a KrF exposure, an EUV exposure, or an electron beam exposure, the sensitivity of the radiation-sensitive resin composition can be further enhanced due to the polymer (A) having the structural unit (II). Therefore, in the case in which the polymer (A) has the structural unit (II), the radiation-sensitive resin composition can be suitably used as a radiation-sensitive resin composition for the KrF exposure, the EUV exposure, or the electron beam exposure.

Examples of the structural unit (II) include a structural unit (hereinafter, may be also referred to as “structural unit (II-1)”) represented by the following formula (II-1), and the like.

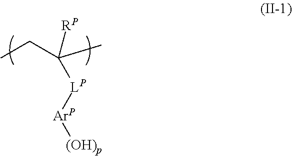

In the above formula (II-1), R^{P} represents a hydrogen atom, a fluorine atom, a methyl group, or a trifluoromethyl group; L^{P} represents a single bond, —COO—, —O—, or —CONH—; Ar¹ represents a group obtained by removing (p+1) hydrogen atoms from a substituted or unsubstituted aromatic hydrocarbon group having 6 to 30 ring atoms; and p is an integer of 1 to 3.

In light of copolymerizability of a monomer that gives the structural unit (II-1), R^{P} represents preferably a hydrogen atom or a methyl group.

L^{P} represents preferably a single bond or —COO—, and more preferably a single bond.

In the case of L^{P} representing the single bond, the resolution resulting from the radiation-sensitive resin composition can be further improved.

Examples of the aromatic hydrocarbon ring structure having 6 to 30 ring atoms which gives Ar^{P} include, of the aromatic ring structures having 5 to 30 ring atoms which gives Ar¹ in the above formula (1), those similar to the ring structures exemplified as the aromatic hydrocarbon ring structure having 6 to 30 ring atoms, and the like. Of these, a benzene structure and a naphthalene structure are preferred, and a benzene structure is more preferred.

A part or all of hydrogen atoms in the aromatic hydrocarbon ring structure may be substituted with a substituent. Examples of the substituent include groups similar to those exemplified as the substituent which may be contained in the ring structure which gives Ar¹, and the like.

p is preferably 1 or 2. In the case in which p is 1, the CDU performance and the resolution resulting from the radiation-sensitive resin composition can be further improved. In the case in which p is 2, the sensitivity of the radiation-sensitive resin composition can be further improved.

Furthermore, in the case in which p is 1, of the carbon atoms constituting Ar^{P}, the hydroxy group preferably bonds to the carbon atom adjacent to the carbon atom which bonds to L^{P}. In the case in which p is no less than 2, of the carbon atoms constituting Ar^{P}, at least one hydroxy group preferably bonds to the carbon atom adjacent to the carbon atom which bonds to L^{P}. In other words, at least one hydroxy group and L^{P} are preferably bonded to Ar^{P} at positions ortho to each other. In this case, generation of defects in the resist pattern formed from the radiation-sensitive resin composition can be inhibited.

Examples of the structural unit (II-1) include structural units represented by the following formulae (II-1-1) to (II-1-18).

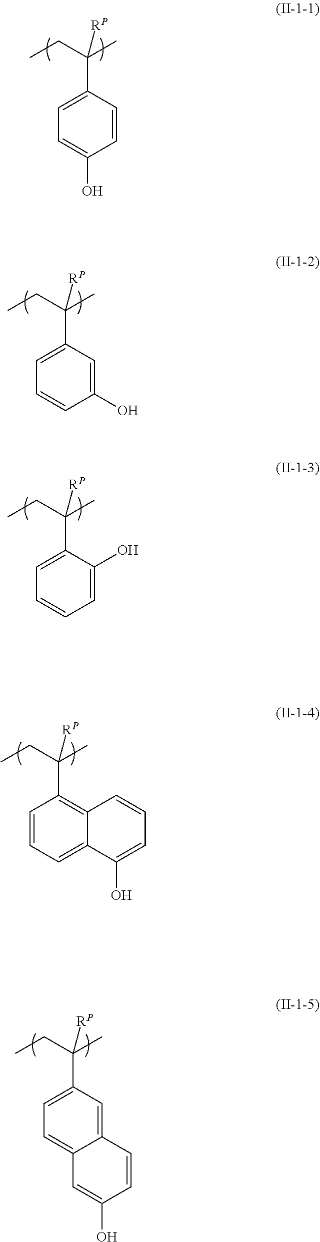

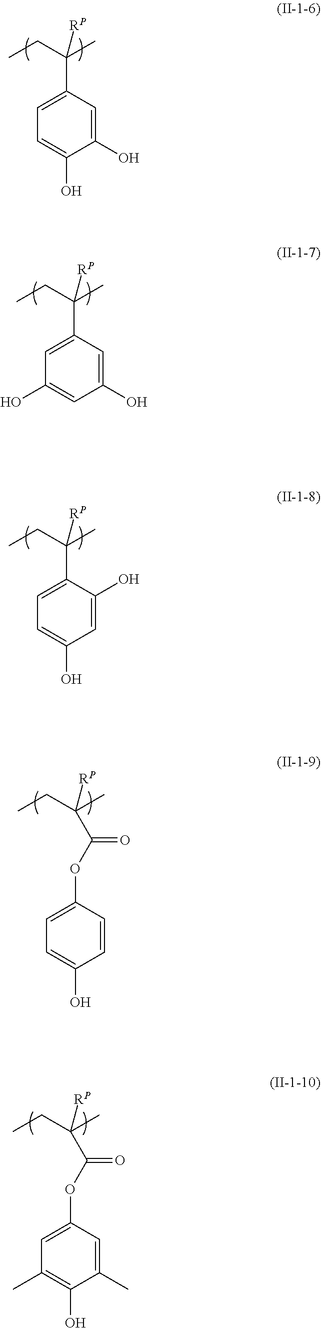

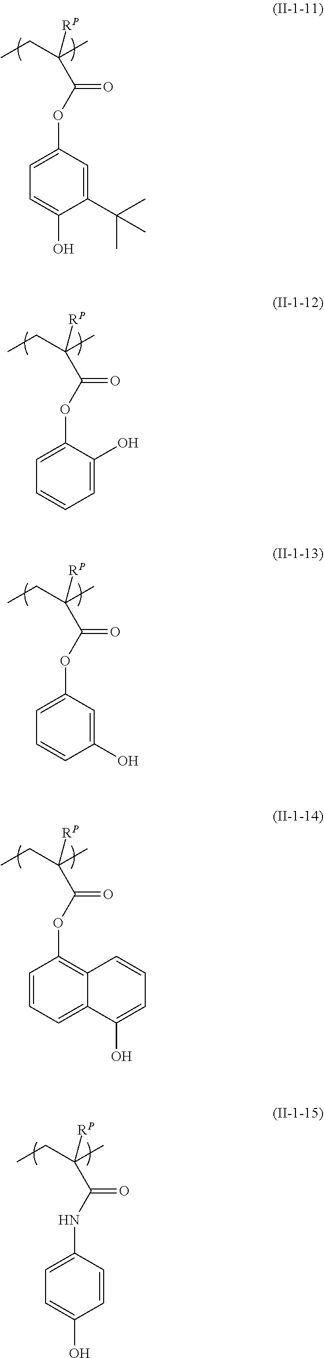

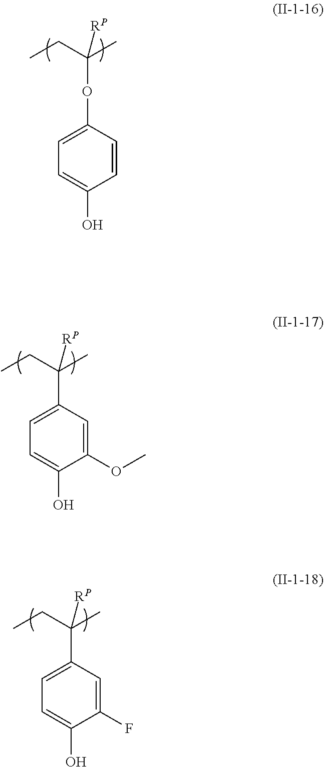

In the above formulae (II-1-1) to (II-1-18), R^{P} is as defined in the above formula (II-1).

In the case in which the polymer (A) has the structural unit (II), the lower limit of a proportion of the structural unit (II) in the polymer (A) with respect to the total structural units constituting the polymer (A) is preferably 10 mol %, and more preferably 20 mol %. The upper limit of the proportion is preferably 60 mol %, and more preferably 50 mol %.

Examples of a monomer which may be also used to give the structural unit (II) include a monomer obtained by substituting a hydrogen atom in a phenolic hydroxy group (—OH) such as 4-acetoxystyrene or 3,5-diacetoxystyrene with an acetyl group or the like. In this case, the polymer (A) having the structural unit (II) can be synthesized by, for example, polymerizing the monomer, and then carrying out a hydrolysis reaction on a polymerization reaction product thus obtained, in the presence of a base such as an amine.

### Structural Unit (III)

The structural unit (III) is a structural unit that includes an acid-labile group (hereinafter, may be also referred to as “(b) acid-labile group” or “acid-labile group (b)”) other than the acid-labile group (a). More specifically, the structural unit (III) is a structural unit including a partial structure obtained by substituting a hydrogen atom in a carboxy group or a phenolic hydroxy group with the acid-labile group (b). The structural unit (III) is a structural unit which is different from the structural unit (I). The polymer (A) may have one, or two or more types of the structural unit (III).

When the polymer (A) has the structural unit (III), the balance of the sensitivity, the CDU performance, and the resolution can be adjusted.

The acid-labile group (b) is a group obtained by substituting a hydrogen atom contained in the carboxy group or the phenolic hydroxy group in the structural unit (III). In other words, the acid-labile group (b) in the structural unit (III) is bonded to the ethereal oxygen atom in the carbonyloxy group or to the oxygen atom in the phenolic hydroxy group.

The acid-labile group (b) is not particularly limited as long as it is a group other than the acid-labile group (a), and examples thereof include groups (hereinafter, may be also referred to as “acid-labile groups (b-1) to (b-3)”) represented by the following formulae (b-1) to (b-3), and the like.

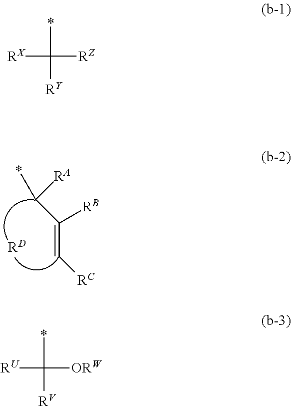

In the above formulae (b-1) to (b-3), * denotes a site bonding to the ethereal oxygen atom in the carbonyloxy group or to the oxygen atom in the phenolic hydroxy group.

In the above formula (b-1), R^{X} represents a substituted or unsubstituted monovalent hydrocarbon group having 1 to 20 carbon atoms; R^{Y} and R^{Z} each independently represent a monovalent hydrocarbon group having 1 to 20 carbon atoms, or R^{Y} and R^{Z} taken together represent a saturated alicyclic structure having 3 to 20 ring atoms, together with the carbon atom to which R^{Y} and R^{Z} bond, wherein in a case in which R^{Y} and R^{Z} represent the hydrocarbon group, candidates are excluded in which: at least one of R^{X}, R^{Y}, and R^{Z} represents a group obtained by removing one hydrogen atom from a substituted or unsubstituted aromatic ring structure having 5 to 30 ring atoms; and two of R^{X}, R^{Y}, and R^{Z} other than the at least one thereof represent a substituted or unsubstituted monovalent aliphatic hydrocarbon group having 1 to 10 carbon atoms.

In the above formula (b-2), R^{A} represents a hydrogen atom; R^{B} and R^{C} each independently represent a hydrogen atom or a monovalent hydrocarbon group having 1 to 20 carbon atoms; and R^{D} represents a divalent hydrocarbon group having 1 to 20 carbon atoms which constitutes an unsaturated alicyclic group having 4 to 20 ring atoms, together with the carbon atom to which each of R^{A}, R^{B}, and R^{C} bonds.

In the above formula (b-3), R^{U} and R^{V} each independently represent a hydrogen atom or a monovalent hydrocarbon group having 1 to 20 carbon atoms, and R^{W} represents a monovalent hydrocarbon group having 1 to 20 carbon atoms, or R^{U} and R^{V} taken together represent an alicyclic structure having 3 to 20 ring atoms together with the carbon atom to which R^{U} and R^{V} bond, or R^{U} and R^{W} taken together represent an aliphatic heterocyclic structure having 4 to 20 ring atoms together with the carbon atom to which R^{U} bonds and the oxygen atom to which R^{W} bonds.

The monovalent hydrocarbon group having 1 to 20 carbon atoms which may be represented by R^{X}, R^{Y}, R^{Z}, R^{B}, R^{C}, R^{U}, R^{V}, or R^{U} is exemplified by a monovalent chain hydrocarbon group having 1 to 20 carbon atoms, a monovalent alicyclic hydrocarbon group having 3 to 20 carbon atoms, a monovalent aromatic hydrocarbon group having 6 to 20 carbon atoms, and the like.

Examples of the monovalent chain hydrocarbon group having 1 to 20 carbon atoms include: alkyl groups such as a methyl group, an ethyl group, an n-propyl group, an i-propyl group, an n-butyl group, a sec-butyl group, an isobutyl group, and a tert-butyl group; alkenyl groups such as an ethenyl group, a propenyl group, a butenyl group, and a 2-methylprop-1-en-1-yl group; alkynyl groups such as an ethynyl group, a propynyl group, and a butynyl group; and the like.

Examples of the monovalent alicyclic hydrocarbon group having 3 to 20 carbon atoms include: monocyclic alicyclic saturated hydrocarbon groups such as a cyclopentyl group and a cyclohexyl group; polycyclic alicyclic saturated hydrocarbon groups such as a norbornyl group, an adamantyl group, a tricyclodecyl group, and a tetracyclododecyl group; monocyclic alicyclic unsaturated hydrocarbon groups such as a cyclopentenyl group and a cyclohexenyl group; polycyclic unsaturated alicyclic hydrocarbon groups such as a norbornenyl group, a tricyclodecenyl group, and a tetracyclododecenyl group; and the like.

Examples of the monovalent aromatic hydrocarbon group having 6 to 20 carbon atoms include: aryl groups such as a phenyl group, a tolyl group, a xylyl group, a naphthyl group, and an anthryl group; aralkyl groups such as a benzyl group, a phenethyl group, a naphthylmethyl group, and an anthrylmethyl group; and the like.

Examples of the substituent which may be contained in the hydrocarbon group represented by R^{X} include groups similar to those exemplified as the substituent which may be contained in the ring structure which gives Ar¹ in the above-described formula (1), and the like.

Examples of the alicyclic structure having 3 to 20 ring atoms which may be represented by R^{Y} and R^{Z} taken together, together with the carbon atom to which R^{Y} and R^{Z} bond, and the alicyclic structure having 3 to 20 ring atoms which may be represented by R^{U} and R^{V} taken together, together with the carbon atom to which R^{U} and R^{V} bond include: monocyclic saturated alicyclic structures such as a cyclopropane structure, a cyclobutane structure, a cyclopentane structure, and a cyclohexane structure; polycyclic saturated alicyclic structures such as a norbornane structure, an adamantane structure, a tricyclodecane structure, and a tetracyclododecane structure; monocyclic unsaturated alicyclic structures such as a cyclopropene structure, a cyclobutene structure, a cyclopentene structure, and a cyclohexene structure; polycyclic unsaturated alicyclic structures such as a norbornene structure, a tricyclodecene structure, and a tetracyclododecene structure; and the like.

Examples of the divalent hydrocarbon group having 1 to 20 carbon atoms represented by R^{D} include groups obtained by removing one hydrogen atom from the groups exemplified as the monovalent hydrocarbon group having 1 to 20 carbon atoms which may be represented by R^{X}, R^{Y}, R^{Z}, R^{B}, R^{C}, R^{U}, R^{V}, or R^{W}, described above; and the like.

Examples of the unsaturated alicyclic ring structure having 4 to 20 ring atoms represented by R^{D}, together with the carbon atom to which each of R^{A}, R^{B}, and R^{C} bond, include: monocyclic unsaturated alicyclic structures such as a cyclobutene structure, a cyclopentene structure, and a cyclohexene structure; polycyclic unsaturated alicyclic structures such as a norbornene structure; and the like.

Examples of the aliphatic heterocyclic structure having 4 to 20 ring atoms which may be represented by R^{U} and R^{W} taken together, together with the carbon atom to which R^{U} bonds and the oxygen atom to which R^{W} bonds, include: saturated oxygen-containing heterocyclic structures such as an oxacyclobutane structure, an oxacyclopentane structure, and an oxacyclohexane structure; unsaturated oxygen-containing heterocyclic structures such as an oxacyclobutene structure, an oxacyclopentene structure, and an oxacyclohexene structure; and the like.

In the case in which each of R^{Y} and R^{Z} represents the monovalent hydrocarbon group having 1 to 20 carbon atoms, each of R^{Y} and R^{Z} is preferably a chain hydrocarbon group, more preferably an alkyl group, and still more preferably a methyl group. R^{X} in this case represents preferably a chain hydrocarbon group, more preferably an alkyl group, and still more preferably a methyl group.

In the case in which R^{Y} and R^{Z} taken together represent the saturated alicyclic structure having 3 to 20 ring atoms together with the carbon atom to which R^{Y} and R^{Z} bond, the saturated alicyclic structure is preferably the monocyclic saturated alicyclic structure, and more preferably a cyclopentane structure. R^{X} in this case represents preferably a chain hydrocarbon group or an aromatic hydrocarbon group, more preferably the alkyl group or a phenyl group, and still more preferably a methyl group, an ethyl group, an i-propyl group, or a phenyl group.

The case in which R^{Y} and R^{Z} taken together represent the saturated alicyclic structure having 3 to 20 ring atoms together with the carbon atom to which R^{Y} and R^{Z} bond is preferred. In this case, the CDU performance resulting from the radiation-sensitive resin composition can be further improved.

R^{B} represents preferably a hydrogen atom.

R^{C} represents preferably a hydrogen atom or a chain hydrocarbon group, more preferably a hydrogen atom or the alkyl group, and still more preferably a methyl group.

The unsaturated alicyclic structure having 4 to 20 ring atoms represented by R^{D}, together with the carbon atom to which each of R^{A}, R^{B}, and R^{C} bond, is preferably a monocyclic unsaturated alicyclic structure, and more preferably a cyclohexene structure.

The acid-labile group (b) is preferably the acid-labile group (b-1) or (b-2).

Examples of the acid-labile group (b-1) include groups represented by the following formulae (b-1-1) to (b-1-5). Examples of the acid-labile group (b-2) include groups represented by the following formula (b-2-1).

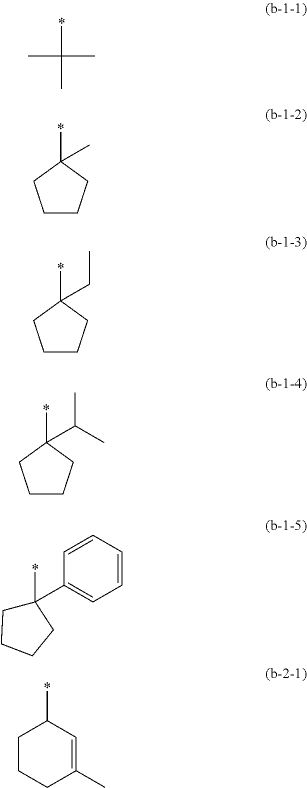

In the above formulae (b-1-1) to (b-1-5) and (b-2-1), * is as defined in the above formulae (b-1) and (b-2).

Examples of the structural unit (III) include a structural unit (hereinafter, may be also referred to as “structural unit (III-1) or (III-2)”) represented by the following formula (III-1) or (III-2), and the like.

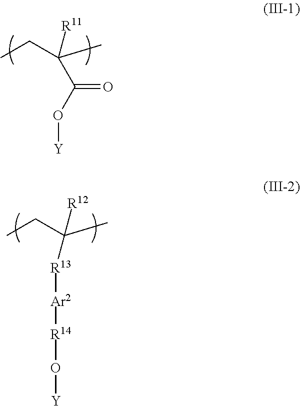

In the above formulae (III-1) and (III-2), Y represents the group (the acid-labile group (b)) represented by the above formulae (b-1) to (b-3).

In the above formula (III-1), R¹¹ is as defined in the above formula (3-1). In the above formula (III-2), R¹², R¹³, Ar², and R¹⁴ are as defined in the above formula (3-2).

The structural unit (III) is preferably the structural unit (III-1).

In the case in which the polymer (A) has the structural unit (III), the lower limit of a proportion of the structural unit (III) in the polymer (A) with respect to the total structural units constituting the polymer (A) is preferably 5 mol %, more preferably 10 mol %, and still more preferably 15 mol %. The upper limit of the proportion is preferably 60 mol %, more preferably 45 mol %, and still more preferably 30 mol %.

### Other Structural Unit(s)

The other structural unit(s) is/are structural unit(s) other than the structural units (I) to (III). The other structural unit(s) is/are exemplified by: a structural unit (hereinafter, may be also referred to as “structural unit (IV)”) that includes a lactone structure, a cyclic carbonate structure, a sultone structure, or a combination thereof; a structural unit (hereinafter, may be also referred to as “structural unit (V)”) that includes an alcoholic hydroxy group; a structural unit (hereinafter, may be also referred to as “structural unit (VI)”) that includes an acid-generating group; and the like.

### Structural Unit (IV)

The structural unit (IV) is a structural unit that includes a lactone structure, a cyclic carbonate structure, a sultone structure, or a combination thereof. When the polymer (A) further has the structural unit (IV), adhesiveness to the substrate can be improved. The polymer (A) may have one, or two or more types of the structural unit (IV).

Examples of the structural unit (IV) include structural units represented by the following formulae, and the like.

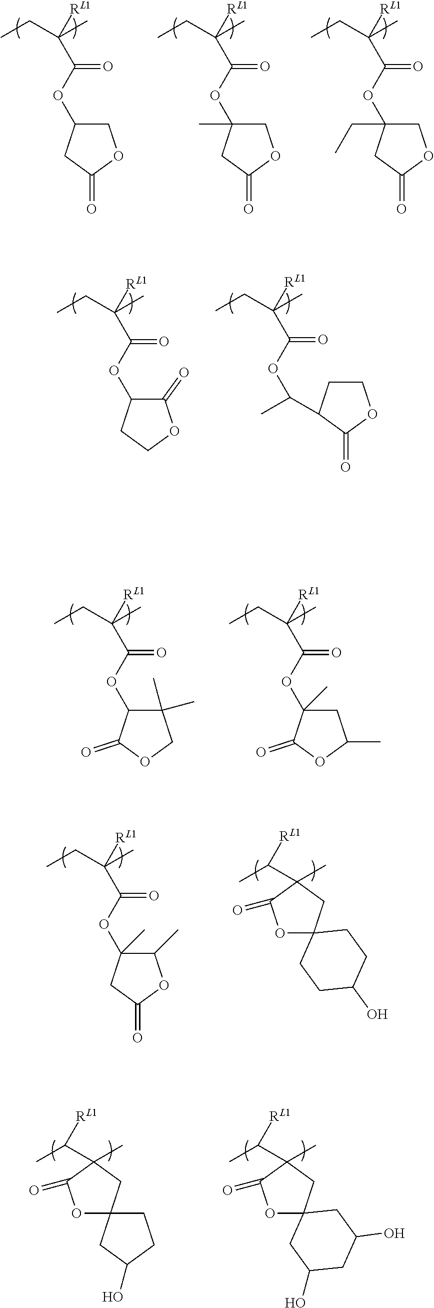

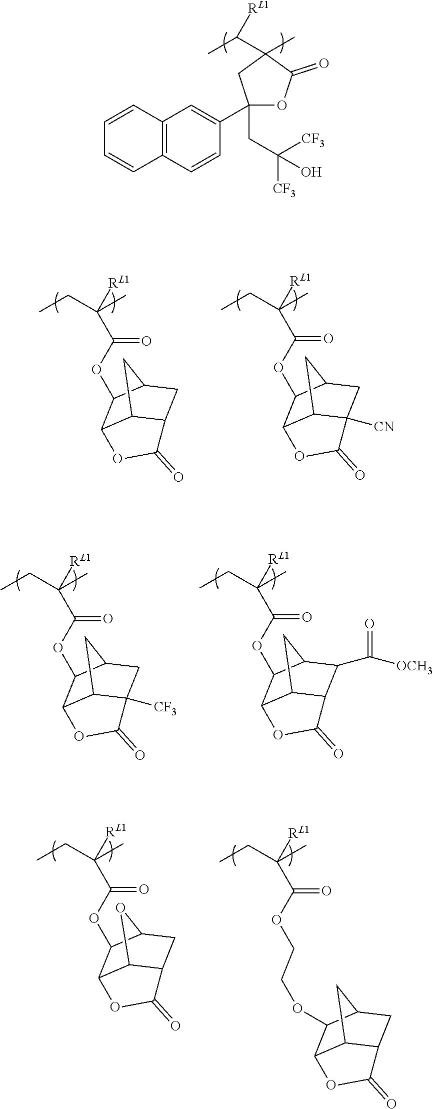

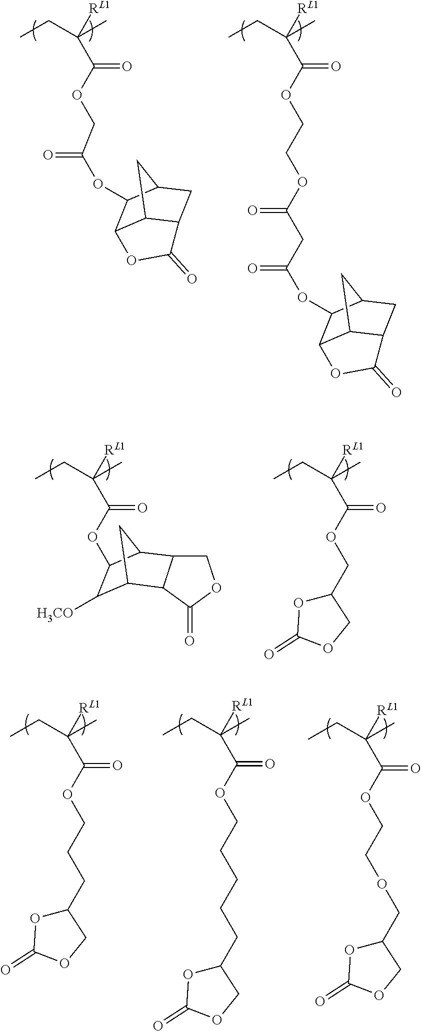

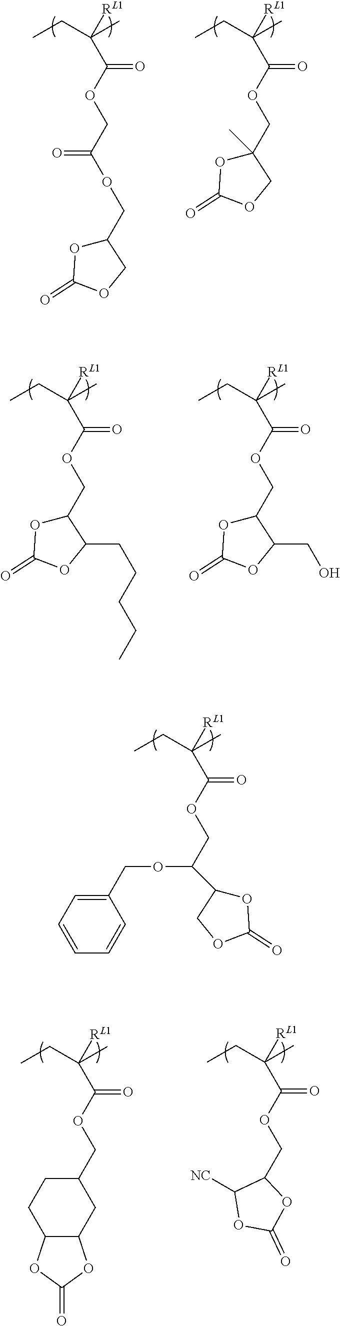

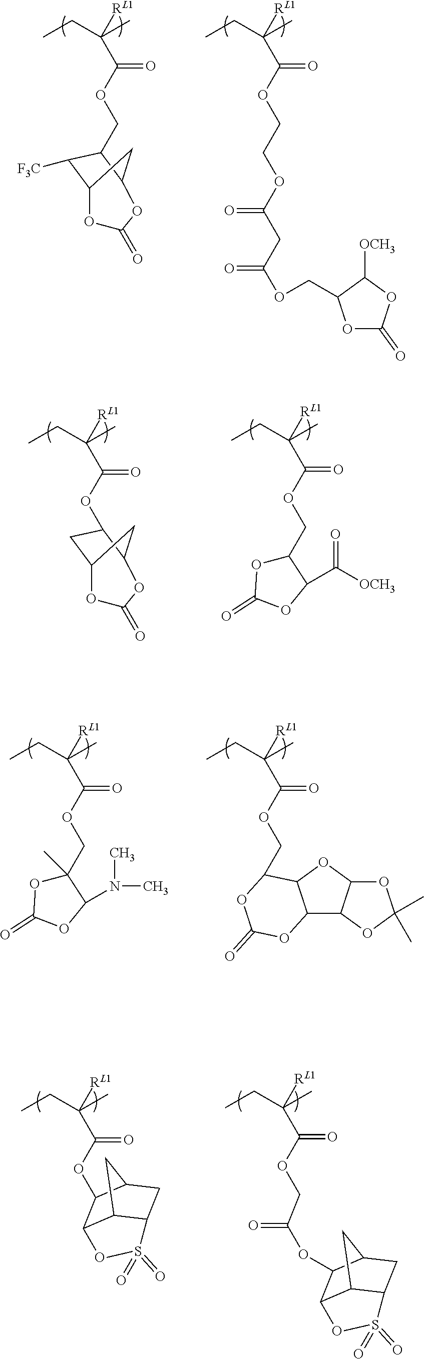

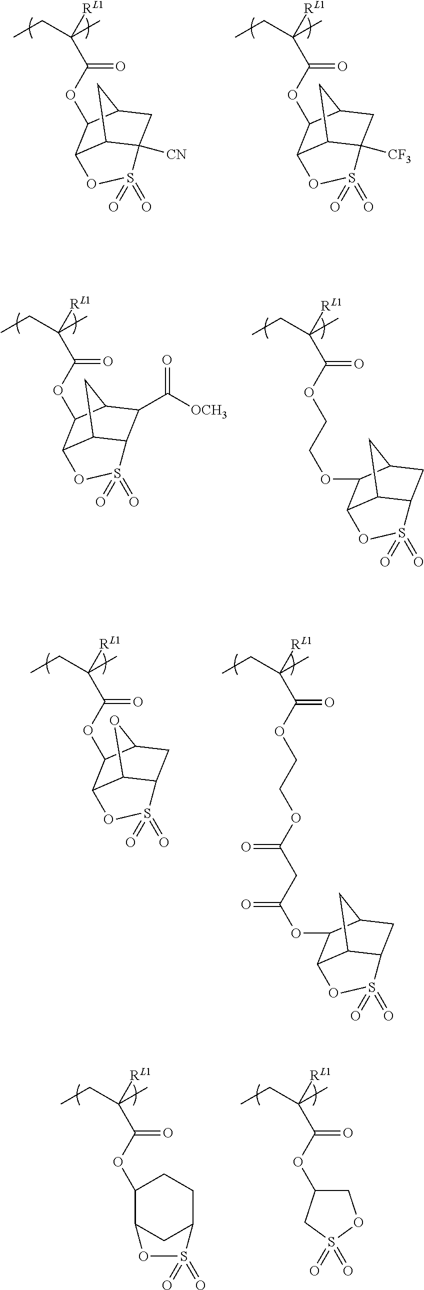

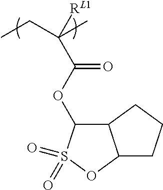

In the above formulae, R^{L1} represents a hydrogen atom, a fluorine atom, a methyl group, or a trifluoromethyl group.

The structural unit (IV) preferably includes a lactone structure.

In the case in which the polymer (A) has the structural unit (IV), the lower limit of a proportion of the structural unit (IV) contained with respect to the total structural units constituting the polymer (A) is preferably 5 mol % and more preferably 10 mol %. The upper limit of the proportion is preferably 35 mol %, and more preferably 25 mol %.

### Structural Unit (V)

The structural unit (V) is a structural unit that includes an alcoholic hydroxy group. When the polymer (A) further has the structural unit (V), the solubility of the polymer (A) in a developer solution can be further appropriately adjusted. The polymer (A) may have one, or two or more types of the structural unit (V).

Examples of the structural unit (V) include structural units represented by the following formulae, and the like.

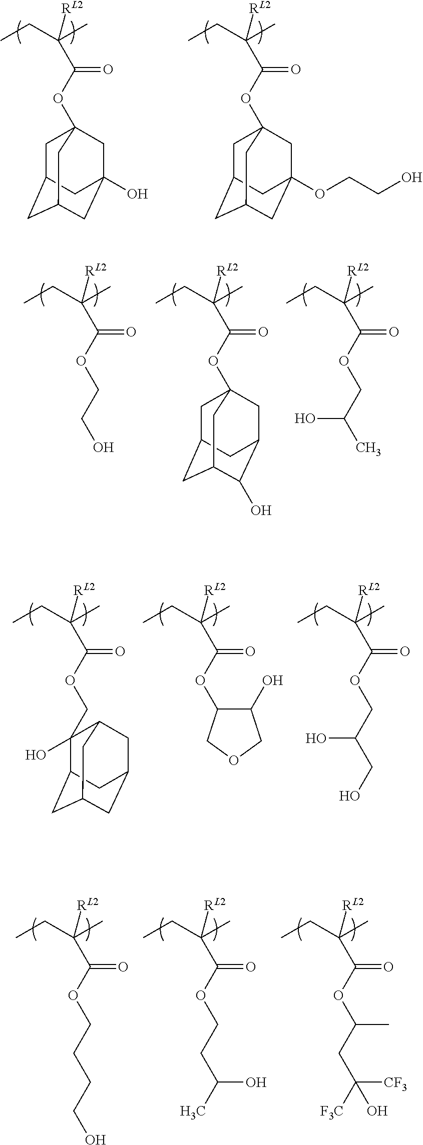

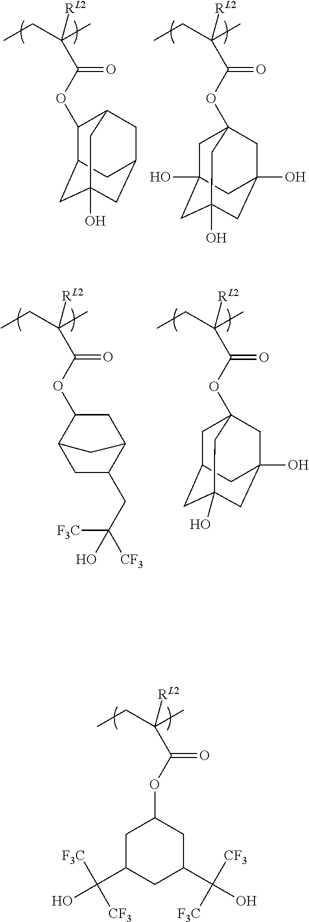

In the above formulae, R^{L2} represents a hydrogen atom, a fluorine atom, a methyl group, or a trifluoromethyl group.

In the case in which the polymer (A) has the structural unit (V), the lower limit of a proportion of the structural unit (V) contained with respect to the total structural units constituting the polymer (A) is preferably 5 mol % and more preferably 10 mol %. The upper limit of the proportion is preferably 35 mol %, and more preferably 25 mol %.

### Structural Unit (VI)

The structural unit (VI) is a structural unit that includes an acid generating group. When the polymer (A) further has the structural unit (VI), the sensitivity to exposure light can be further enhanced. The polymer (A) may include one, or two or more types of the structural unit (VI).

The acid generating group is a group that generates an acid upon irradiation with a radioactive ray. The structural unit (VI) is exemplified by a structural unit derived from a monomer constituted from an organic acid anion including a polymerizable group and a radiation-sensitive onium cation, a structural unit derived from a monomer constituted from a radiation-sensitive onium cation including an organic acid anion and a polymerizable group, and the like. Examples of the organic acid anion include a sulfonate anion and a carboxylate anion. Examples of the radiation-sensitive onium cation include a radiation-sensitive onium cation contained in: the compound (Z), the acid generating agent (B), or the acid diffusion control agent (C), each described later.

The structural unit (VI) preferably includes an iodo group in addition to the acid-generating group. Accordingly, the sensitivity of the radiation-sensitive resin composition may be further improved. The iodo group may be contained in the organic acid anion, or may be contained in the radiation-sensitive onium cation.

In the case in which the polymer (A) has the structural unit (VI), the lower limit of a proportion of the structural unit (VI) contained with respect to the total structural units constituting the polymer (A) is preferably 1 mol % and more preferably 5 mol %. The upper limit of the proportion is preferably 20 mol %, and more preferably 15 mol %.

### (Z) Compound

The compound (Z) is a compound having: a monovalent radiation-sensitive onium cation moiety (hereinafter, may be also referred to as “cation moiety (P)”) including an aromatic ring structure obtained by substituting at least one hydrogen atom with a fluorine atom or a fluorine atom-containing group; and a monovalent organic acid anion moiety (hereinafter, may be also referred to as “anion moiety (Q)”). The radiation-sensitive resin composition may contain one, or two or more types of the compound (Z).

Depending on the type of the anion group included in the anion moiety (Q), the compound (Z) has: an effect of generating an acid upon irradiation with a radioactive ray in the radiation-sensitive resin composition; or an effect of inhibiting an undesired chemical reaction (for example, a dissociation reaction of the acid-labile group) in light-unexposed regions by controlling a phenomenon in which an acid generated from the acid generating agent (B) and/or the like upon exposure is diffused in the resist film. In other words, depending on the type of the anion group, the compound (Z) functions as a radiation-sensitive acid generating agent or an acid diffusion control agent (quencher) in the radiation-sensitive resin composition.

In the case in which the compound (Z) functions as the radiation-sensitive acid generating agent, the radioactive ray may be exemplified by radioactive rays similar to those exemplified as the exposure light in the exposing in the method of forming a resist pattern of an other embodiment of the present invention, described later. The acid-labile group (a) included in the structural unit (I) which is contained in the polymer (A), or the like is dissociated by an action of an acid generated from the compound (Z) upon irradiation with the radioactive ray, whereby a carboxy group, a phenolic hydroxy group, and/or the like are/is generated to create a difference in solubility of the resist film in the developer solution between light-exposed regions and light-unexposed regions; accordingly, a resist pattern can be formed.

In the case in which the compound (Z) functions as the acid diffusion control agent, an acid is generated in the light-exposed regions to increase the solubility or insolubility of the polymer (A) in the developer solution, and a superior acid-trapping function by the anion is exhibited, functioning as a quencher in the light-unexposed regions, whereby acid diffused from the light-exposed regions is trapped. Thus, the compound (Z) can enhance roughness at interfaces between the light-exposed regions and the light-unexposed regions, and improve the resolution by enhancing the contrast between the light-exposed regions and the light-unexposed regions.

Despite the aforementioned effect of the compound (Z) in the radiation-sensitive resin composition, described above, it is considered that containing the compound (Z) in the radiation-sensitive resin composition is a factor in the radiation-sensitive resin composition exhibiting superior sensitivity, and resulting in superior CDU performance and resolution. Although not necessarily clarified and without wishing to be bound by any theory, it is presumed that the reason for this is that due to the compound (Z) having the cation moiety (P), the amount of acid generated is improved.

In the case in which the compound (Z) functions as the radiation-sensitive acid generating agent, the lower limit of a content of the compound (Z) in the radiation-sensitive resin composition with respect to 100 parts by mass of the polymer (A) is preferably 10 parts by mass, more preferably 20 parts by mass, and still more preferably 30 parts by mass. The upper limit of the content is preferably 80 parts by mass, more preferably 70 parts by mass, and still more preferably 60 parts by mass.

In the case in which the compound (Z) functions as the acid diffusion control agent, the lower limit of a proportion of the compound (Z) in the radiation-sensitive resin composition acid with respect to 100 mol % of the radiation-sensitive acid generating agent (the compound (Z) functioning as the radiation-sensitive acid generating agent, and/or the acid generating agent (B)) contained in the radiation-sensitive resin composition is preferably 10 mol %, more preferably 20 mol %, and still more preferably 30 mol %. The upper limit of the proportion is preferably 90 mol %, more preferably 80 mol %, and still more preferably 70 mol %.

Each structure contained in the compound (Z) is described below.

### Cation Moiety (P)

The cation moiety (P) is a monovalent radiation-sensitive onium cation. The cation moiety (P) includes an aromatic ring structure (hereinafter, may be also referred to as “aromatic ring structure (p)”) obtained by substituting at least one hydrogen atom with a fluorine atom or a fluorine atom-containing group. It is considered that the cation moiety (P) including the aromatic ring structure (p) is a factor in the radiation-sensitive resin composition exhibiting superior sensitivity, and resulting in superior CDU performance and resolution.

Examples of the aromatic ring structure which gives the aromatic ring structure (p) include structures similar to those exemplified as the aromatic ring structure having 5 to 30 ring atoms which gives Ar¹ in the above formula (1), and the like. Of these, the aromatic hydrocarbon ring structure having 6 to 30 ring atoms or the aromatic heterocyclic structure having 6 to 30 ring atoms is preferred, a benzene structure, a fused polycyclic aromatic hydrocarbon ring structure, or a sulfur atom-containing heterocyclic structure is more preferred, and a benzene structure, a naphthalene structure, or a dibenzothiophene structure is still more preferred.

In the aromatic ring structure (p), at least one hydrogen atom which bonds to an atom constituting an aromatic ring is substituted with a fluorine atom or a fluorine atom-containing group. The “fluorine atom-containing group” as referred to means a group having at least one fluorine atom. Examples of the fluorine atom-containing group include a group (hereinafter, may be also referred to as “fluorinated hydrocarbon group”) obtained by substituting a part or all of hydrogen atoms in a monovalent hydrocarbon group having 1 to 20 carbon atoms with a fluorine atom, and the like. The fluorine atom-containing group is preferably a fluorinated alkyl group, and more preferably a trifluoromethyl group.

The number of substitutions with a fluorine atom or a fluorine atom-containing group in the aromatic ring structure (p) is no less than 1. The number of substitutions is preferably 1 to 3, and more preferably 1 or 2.

Furthermore, substitution of a hydrogen atom which bonds to an atom constituting the aromatic ring structure (p) may be carried out with a substituent other than a fluorine atom or a fluorine atom-containing group. Examples of such a substituent include substituents other than those corresponding to a fluorine atom or a fluorine atom-containing group among those exemplified as the substituent which may be contained in the ring structure that gives Ar¹. The substituent is preferably an alkyl group, an iodine atom, or a bromine atom, and more preferably a methyl group or an iodine atom.

Examples of a cation species in the cation moiety (P) include a sulfonium cation (S⁺), an iodonium cation (I⁺), and the like. Of these, the sulfonium cation is preferred.

The cation moiety (P) includes at least one aromatic ring structure (p). The cation moiety (P) may include an aromatic ring structure other than the aromatic ring structure (p). In the case in which the cation species of the cation moiety (P) is a sulfonium cation, the cation moiety (P) is classified broadly into: a form (form 1) which includes three aromatic ring structures; and a form (form 2) which includes one aromatic ring structure and one ring structure which contains a sulfur atom of a sulfonium cation as a ring-constituting atom. In the case of form 1, the cation moiety (P) preferably includes at least two aromatic ring structures (p). Examples of the ring structure containing the sulfur atom of the sulfonium cation as the ring-constituting atom include a benzothiophene structure, a dibenzothiophene structure, and the like.

The cation moiety (P) is preferably a cation (hereinafter, may be also referred to as “cation (P-1) or (P-2)”) represented by the following formula (2-1) or (2-2).

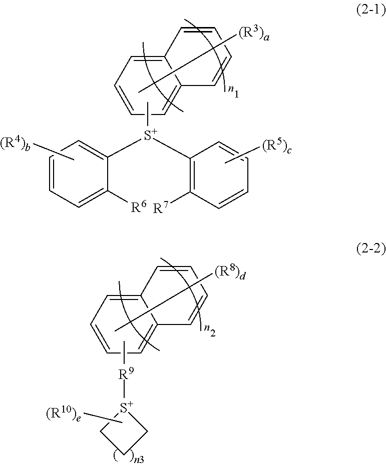

In the formula (2-1), a is an integer of 0 to 7, b is an integer of 0 to 4, and c is an integer of 0 to 4, wherein a sum of a, b, and c is no less than 1; R³, R⁴, and R⁵ each independently represent a halogen atom, a hydroxy group, a nitro group, or a monovalent organic group having 1 to 20 carbon atoms, wherein at least one of R³, R⁴, and R⁵ represents a fluorine atom or a monovalent fluorinated hydrocarbon group having 1 to 10 carbon atoms; in a case in which a is no less than 2, a plurality of R³s are identical or different from each other, in a case in which b is no less than 2, a plurality of R⁴s are identical or different from each other, and in a case in which c is no less than 2, a plurality of R⁵ s are identical or different from each other; R⁶ and R⁷ each independently represent a hydrogen atom, a fluorine atom, or a monovalent fluorinated hydrocarbon group having 1 to 10 carbon atoms, or R⁶ and R⁷ taken together represent a single bond; and n₁ is 0 or 1.

In the formula (2-2), d is an integer of 1 to 7 and e is an integer of 0 to 10, wherein in a case in which d is 1, R⁸ represents a fluorine atom or a monovalent fluorinated hydrocarbon group having 1 to 10 carbon atoms, and in a case in which d is no less than 2, a plurality of R⁸s are identical or different from each other, and each R⁸ represents a halogen atom, a hydroxy group, a nitro group, or a monovalent organic group having 1 to 20 carbon atoms, wherein at least one of the plurality of R⁸s represents a fluorine atom or a monovalent fluorinated hydrocarbon group having 1 to 10 carbon atoms; R⁹ represents a single bond or a divalent organic group having 1 to 20 carbon atoms; R¹⁰ represents a halogen atom, a hydroxy group, a nitro group, or a monovalent organic group having 1 to 20 carbon atoms, wherein in a case in which e is no less than 2, a plurality of R¹⁰s are identical or different from each other; n₂ is 0 or 1; and n 3 is an integer of 0 to 3.

The “organic group” as referred to herein means a group that includes at least one carbon atom.

The monovalent organic group having 1 to 20 carbon atoms is exemplified by: a monovalent hydrocarbon group having 1 to 20 carbon atoms; a group (hereinafter, may be also referred to as “group (α)”) that contains a divalent heteroatom-containing group between two adjacent carbon atoms of the monovalent hydrocarbon group; a group (hereinafter, may be also referred to as “group (β)”) obtained by substituting with a monovalent heteroatom-containing group, a part or all of hydrogen atoms included in the monovalent hydrocarbon group or the group (α); a group (hereinafter, may be also referred to as “group (γ)”) obtained by combining the monovalent hydrocarbon group, the group (α), or the group (β) with a divalent heteroatom-containing group; and the like.

Exemplary heteroatoms which may constitute the divalent or monovalent heteroatom-containing group include an oxygen atom, a nitrogen atom, a sulfur atom, a phosphorus atom, a silicon atom, a halogen atom, and the like.

Examples of the monovalent heteroatom-containing group include a halogen atom, a hydroxy group, a carboxy group, a cyano group, an amino group, a sulfanyl group (—SH), an oxo group (═O), and the like.

The divalent heteroatom-containing group is exemplified by —O—, —CO—, —S—, —CS—, —NR′—, groups obtained by combination of two or more of these (for example, —COO—, —CONR′—, etc.), and the like. R′ represents a hydrogen atom or a monovalent hydrocarbon group having 1 to 10 carbon atoms. Examples of the monovalent hydrocarbon group having 1 to 10 carbon atoms represented by R′ include, among the groups exemplified above as the “monovalent hydrocarbon group having 1 to 20 carbon atoms”, those having 1 to 10 carbon atoms, and the like.

The halogen atom is a fluorine atom, a chlorine atom, a bromine atom, or an iodine atom.

The monovalent fluorinated hydrocarbon group having 1 to 10 carbon atoms is a group obtained by substituting a part or all of hydrogen atoms in a monovalent hydrocarbon group having 1 to 10 carbon atoms with a fluorine atom. Specific examples include fluorinated alkyl groups, e.g., partially fluorinated alkyl groups such as a fluoromethyl group, a difluoromethyl group, a difluoroethyl group, a trifluoroethyl group, and a trifluoropropyl group; perfluoroalkyl groups such as a trifluoromethyl group, a pentafluoroethyl group, and a hexafluoropropyl group; and the like. Of these, the perfluoroalkyl groups are preferred, and a trifluoromethyl group is more preferred.

Examples of the divalent organic group having 1 to 20 carbon atoms include a group obtained by removing one hydrogen atom from the monovalent organic group having 1 to 20 carbon atoms, and the like.

A sum of a, b, and c is preferably 1 to 6, and more preferably 3 to 5. a, b, and c may be appropriately selected from within this range.

It is preferred that R⁶ and R⁷ each represent a hydrogen atom, or that R⁶ and R⁷ taken together represent a single bond.

The cation moiety (P) is preferably the cation (P-1).

Examples of the cation (P-1) include cations represented by the following formulae (P-1-1) to (P-1-11), and the like.

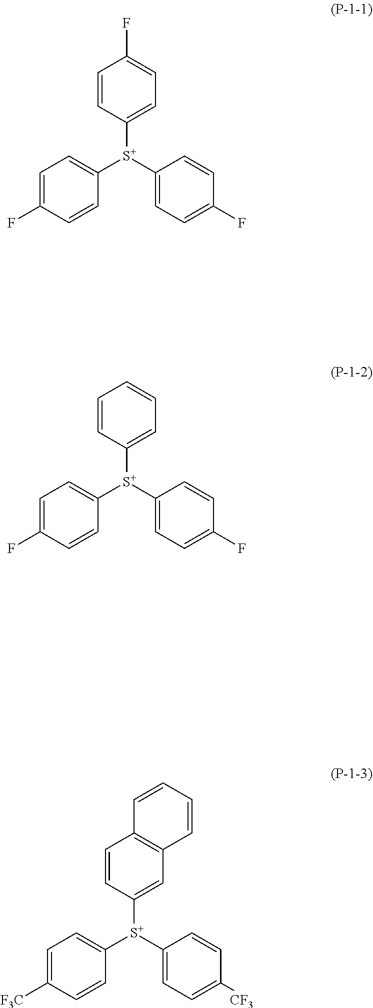 [SMILES: Fc1ccc([S+](c2ccc(F)cc2)c2ccc(F)cc2)cc1 ; Fc1ccc([S+](c2ccccc2)c2ccc(F)cc2)cc1 ; FC(F)(F)c1ccc([S+](c2ccc(C(F)(F)F)cc2)c2ccc3ccccc3c2)cc1]

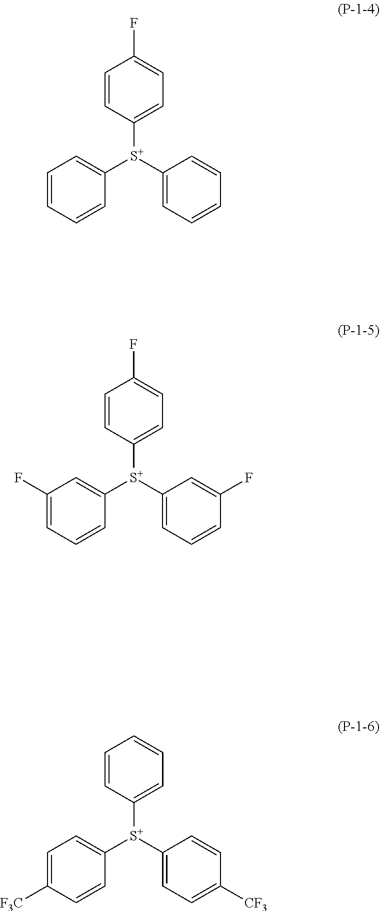 [SMILES: Fc1ccc([S+](c2ccccc2)c2ccccc2)cc1 ; Fc1ccc([S+](c2cccc(F)c2)c2cccc(F)c2)cc1 ; FC(F)(F)c1ccc([S+](c2ccccc2)c2ccc(C(F)(F)F)cc2)cc1]

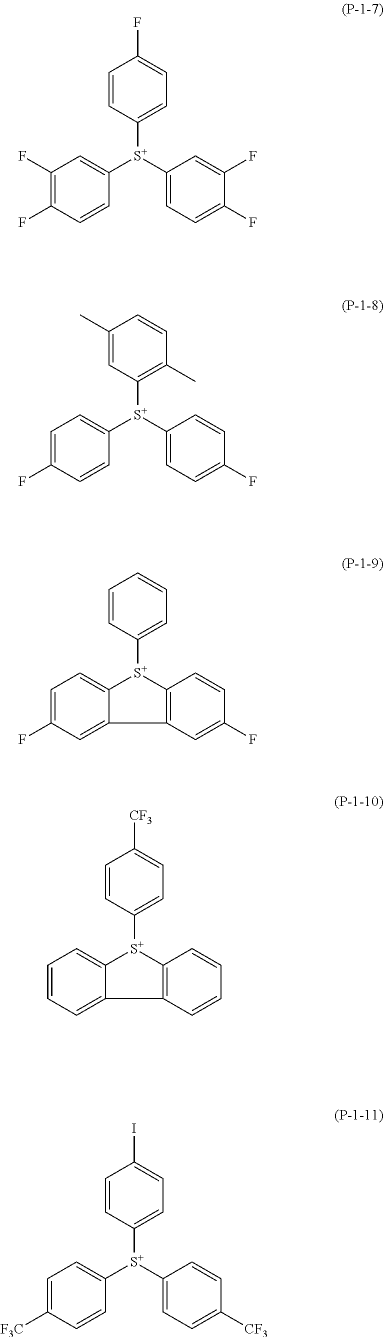 [SMILES: Fc1ccc([S+](c2ccc(F)c(F)c2)c2ccc(F)c(F)c2)cc1 ; Cc1ccc(C)c([S+](c2ccc(F)cc2)c2ccc(F)cc2)c1 ; Fc1ccc2c(c1)c1cc(F)ccc1[s+]2-c1ccccc1 ; FC(F)(F)c1ccc(-[s+]2c3ccccc3c3ccccc32)cc1 ; FC(F)(F)c1ccc([S+](c2ccc(I)cc2)c2ccc(C(F)(F)F)cc2)cc1]

### Anion Moiety (Q)

The anion moiety (Q) is a monovalent organic acid anion. The anion moiety (Q) includes a monovalent anion group. Examples of the monovalent anion group include a sulfonate anion group (—SO₃), a carboxylate anion group (—COO), a sulfonimidate anion group (—SO₂—N″—SO₂—), and the like. Of these, the sulfonate anion group or the carboxylate anion group is preferred.

Hereinafter, among the anion moieties (Q), one having a sulfonate anion group as the monovalent anion group is referred to as “anion moiety (Q-1)”, and one having a carboxylate anion group as the monovalent anion group is referred to as “anion moiety (Q-2)”.

### Anion Moiety (Q-1)

In the case in which the compound (Z) has the anion moiety (Q-1), the compound (Z) functions as the radiation-sensitive acid generating agent or the acid diffusion control agent. In the case in which the compound (Z) functions as the radiation-sensitive acid generating agent, the radiation-sensitive resin composition preferably contains an acid diffusion control agent. Examples of the acid diffusion control agent include the compound (Z) in the case of functioning as the acid diffusion control agent, the acid diffusion control agent (C), described later, and the like. Of these, the acid diffusion control agent is preferably the compound (Z) in the case of functioning as the acid diffusion control agent. In other words, the radiation-sensitive resin composition preferably contains the compound (Z) having the anion moiety (Q-1), and the compound (Z) having the anion moiety (Q-2). In this case, the CDU performance resulting from the radiation-sensitive resin composition can be further improved.

The anion moiety (Q-1) is not particularly limited as long as it is used as an anion moiety in an onium salt-type radiation-sensitive acid generating agent, and examples thereof include an sulfonate anion represented by the following formula (4-1).

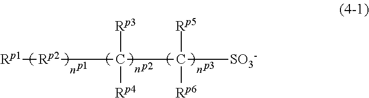

In the above formula (4-1), R^{p1} represents a monovalent group containing a ring structure having 5 or more ring atoms; R^{p2} represents a divalent linking group; R^{p3} and R^{p4} each independently represent a hydrogen atom, a fluorine atom, a monovalent hydrocarbon group having 1 to 20 carbon atoms, or a monovalent fluorinated hydrocarbon group having 1 to 20 carbon atoms; R^{p5} and R^{p6} each independently represent a fluorine atom or a monovalent fluorinated hydrocarbon group having 1 to 20 carbon atoms; n^{p1} is an integer of 0 to 10, n^{p2} is an integer of 0 to 10, and n^{p3} is an integer of 0 to 10, wherein a sum of n^{p1}, n^{p2}, and n^{p3} is no less than 1 and no greater than 30, in a case in which n^{p1} is no less than 2, a plurality of R^{p2}s are identical to or different from each other, in a case in which n^{p2} is no less than 2, a plurality of R^{p3}s are identical to or different from each other and a plurality of R^{p4}s are identical to or different from each other, and in a case in which n^{p3} is no less than 2, a plurality of R^{p5}s are identical to or different from each other and a plurality of R^{p6}s are identical to or different from each other.

The ring structure having 5 or more ring atoms is exemplified by an aliphatic hydrocarbon ring structure having 5 or more ring atoms, an aliphatic heterocyclic structure having 5 or more ring atoms, an aromatic hydrocarbon ring structure having 6 or more ring atoms, an aromatic heterocyclic structure having 5 or more ring atoms, or a combination thereof.

Examples of the aliphatic hydrocarbon ring structure having 5 or more ring atoms include: monocyclic saturated alicyclic structures such as a cyclopentane structure, a cyclohexane structure, a cycloheptane structure, a cyclooctane structure, a cyclononane structure, a cyclodecane structure and a cyclododecane structure; monocyclic unsaturated alicyclic structures such as a cyclopentene structure, a cyclohexene structure, a cycloheptene structure, a cyclooctene structure and a cyclodecene structure; polycyclic saturated alicyclic structures such as a norbornane structure, an adamantane structure, a tricyclodecane structure and a tetracyclododecane structure, and a steroid structure; polycyclic unsaturated alicyclic structures such as a norbornene structure and a tricyclodecene structure; and the like. The “steroid structure” as referred to means a structure having, as a basic skeleton, a skeleton (sterane skeleton) in which three 6-membered rings and one 4-membered ring are fused.

Examples of the aliphatic heterocyclic structure having 5 or more ring atoms include: lactone structures such as a hexanolactone structure and a norbornanelactone structure; sultone structures such as a hexanosultone structure and a norbornanesultone structure; oxygen atom-containing heterocyclic structures such as a dioxolane structure, an oxacycloheptane structure, and an oxanorbornane structure; nitrogen atom-containing heterocyclic structures such as an azacyclohexane structure and a diazabicyclooctane structure; sulfur atom-containing heterocyclic structures such as a thiacyclohexane structure and a thianorbornane structure; and the like.

Examples of the aromatic hydrocarbon ring structure having 6 or more ring atoms include: a benzene structure; fused polycyclic aromatic hydrocarbon ring structures such a naphthalene structure, an anthracene structure, a fluorene structure, biphenylene structure, a phenanthrene structure, and a pyrene structure; ring-assembled aromatic hydrocarbon ring structures such as a biphenyl structure, a terphenyl structure, a binaphthalene structure, and a phenylnaphthalene structure; a 9,10-ethanoanthracene structure; and the like.

Examples of the aromatic heterocyclic structure having 5 or more ring atoms include: oxygen atom-containing heterocyclic structures such as a furan structure, a pyran structure, a benzofuran structure, and a benzopyran structure; nitrogen atom-containing heterocyclic structures such as a pyridine structure, a pyrimidine structure, and an indole structure; sulfur atom-containing heterocyclic structures such as a thiophene structure; and the like.

In the ring structure, a part or all of hydrogen atoms which bond to an atom constituting the ring structure may be substituted with a substituent. Examples of the substituent include groups similar to those exemplified as the substituent which may be contained in the ring structure which gives Ar¹ in the above-described formula (1), and the like. The substituent is preferably an iodine atom, an alkyl group, or an alkoxy group.

The lower limit of the number of ring atoms of the ring structure is preferably 6, more preferably 8, still more preferably 9, and particularly preferably 10. The upper limit of the number of ring atoms is preferably 25.

R^{p1} represents preferably a monovalent group including the aliphatic hydrocarbon ring structure having 5 or more ring atoms, a monovalent group including the aliphatic heterocyclic structure having 5 or more ring atoms, or the aromatic hydrocarbon ring structure having 6 or more ring atoms.

Examples of the divalent linking group which may be represented by R^{p2} include a carbonyl group, an ether group, a carbonyloxy group, a sulfide group, a thiocarbonyl group, a sulfonyl group, a divalent hydrocarbon group, a combination thereof, and the like.

The monovalent hydrocarbon group having 1 to 20 carbon atoms which may be represented by each of R^{p3} and R^{p4} is exemplified by an alkyl group having 1 to 20 carbon atoms, and the like. The monovalent fluorinated hydrocarbon group having 1 to 20 carbon atoms which may be represented by each of R^{p3} and R^{p4} is exemplified by a fluorinated alkyl group having 1 to 20 carbon atoms, and the like. R^{p3} and R^{p6} each represent preferably a hydrogen atom, a fluorine atom, or a fluorinated alkyl group, more preferably a hydrogen atom, a fluorine atom, or a perfluoroalkyl group, and still more preferably a hydrogen atom, a fluorine atom, or a trifluoromethyl group.

The monovalent fluorinated hydrocarbon group having 1 to 20 carbon atoms which may be represented by each of R^{p5} and R^{p6} is exemplified by a fluorinated alkyl group having 1 to 20 carbon atoms, and the like. R^{p5} and R^{p6} each independently represent preferably a fluorine atom or a fluorinated alkyl group, more preferably a fluorine atom or a perfluoroalkyl group, still more preferably a fluorine atom or a trifluoromethyl group, and particularly preferably a fluorine atom.

n^{p1} is preferably 0 to 5, more preferably 0 to 2, and still more preferably 0 or 1.

n^{p2} is preferably 0 to 5, more preferably 0 to 2, and still more preferably 0 or 1.

The lower limit of n^{p3} is preferably 1, and more preferably 2. When n^{p3} is no less than 1, strength of the acid can be enhanced. The upper limit of n^{p3} is preferably 4, more preferably 3, and still more preferably 2.

The lower limit of the sum of n^{p1}, n^{p2}, and n^{p3} is preferably 2, and more preferably 4. The upper limit of the sum of n^{p1}, n^{p2}, and n^{p3} is preferably 20, and more preferably 10.

The anion moiety (Q-1) is preferably a sulfonate anion represented by the following formulae (4-1-1) to (4-1-12).

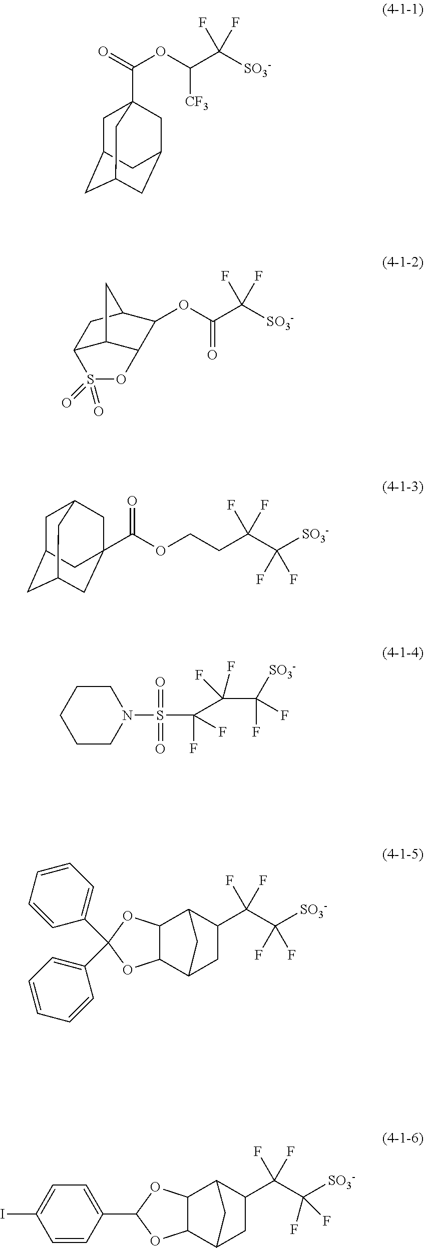

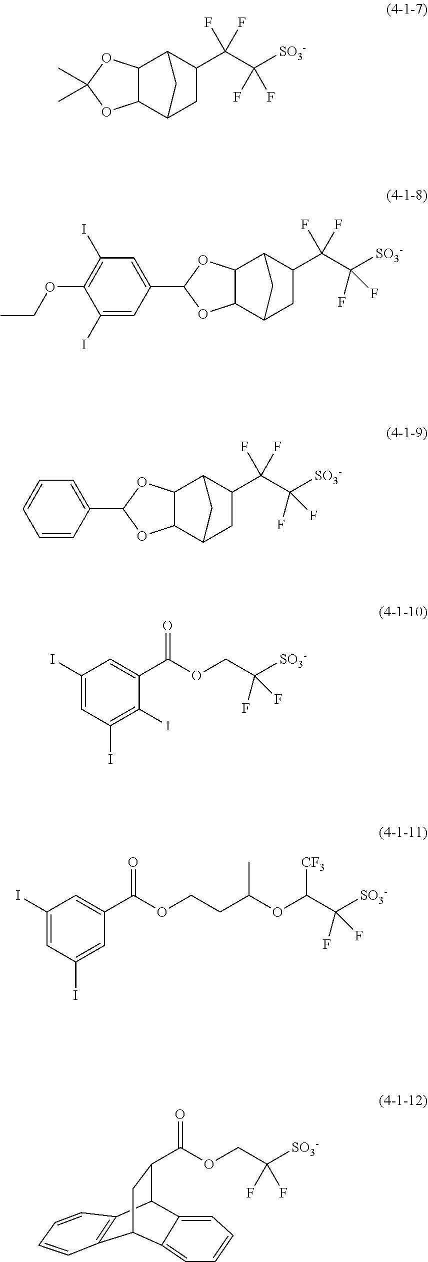

The compound (Z) which may be used as the radiation-sensitive acid generating agent is a compound obtained by appropriately combining the cation moiety (P) and the anion moiety (Q-1).

### Anion Moiety (Q-2)

In the case in which the compound (Z) has the anion moiety (Q-2), the compound (Z) functions as the acid diffusion control agent. In this case, the radiation-sensitive resin composition preferably has a radiation-sensitive acid generating agent. Examples of the radiation-sensitive acid generating agent include the compound (Z) in the case of functioning as the radiation-sensitive acid generating agent, the acid-generating agent (B), described later, and the like. Of these, the radiation-sensitive acid generating agent is preferably the compound (Z) in the case of functioning as the radiation-sensitive acid generating agent.

The anion moiety (Q-2) is not particularly limited as long as it is used as an anion moiety in a photodegradable base that is photosensitized by exposure to generate a weak acid, and examples thereof include a substituted or unsubstituted salicylate anion, a group obtained by replacing the sulfonate anion group in the above formula (4-1) with a carboxylate anion group, and the like.

Preferable examples of the anion moiety (Q-2) include carboxylate anions represented by the following formulae (4-2-1) to (4-2-8).

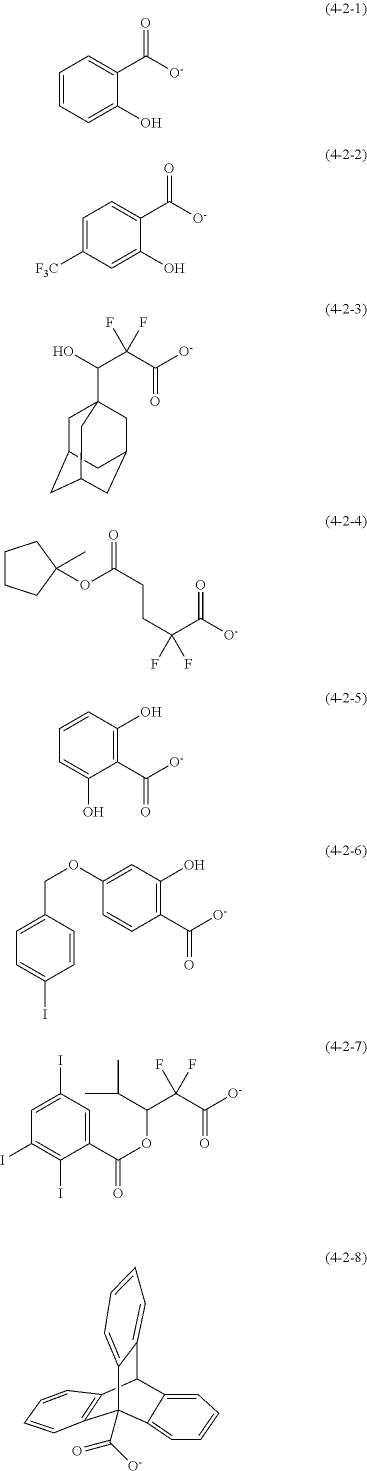

The compound (Z) which may be used as the acid diffusion control agent is a compound obtained by appropriately combining the cation moiety (P) and the anion moiety (Q-2).

### (B) Acid Generating Agent

The acid generating agent (B) is a radiation-sensitive acid generating agent other than the compound (Z) as the radiation-sensitive acid generating agent. The acid generating agent (B) is exemplified by an onium salt compound, an N-sulfonyloxyimide compound, a sulfonimide compound, a halogen-containing compound, a diazoketone compound, and the like.

Examples of the onium salt compound include a sulfonium salt, a tetrahydrothiophenium salt, an iodonium salt, a phosphonium salt, a diazonium salt, a pyridinium salt, and the like. With regard to this, the cation moiety in this case does not encompass an aromatic ring structure obtained by substituting a hydrogen atom with a fluorine atom or a fluorine atom-containing group.

Examples of the acid generating agent (B) include a compound obtained by combining a substituted or unsubstituted triphenylsulfonium cation and the anion moiety (Q-1) described in the above section “(Z) Compound”, and the like.

In the case in which the radiation-sensitive resin composition contains the acid generating agent (B), the lower limit of a content of the acid generating agent (B) in the radiation-sensitive resin composition with respect to 100 parts by mass of the polymer (A) is preferably 10 parts by mass, more preferably 20 parts by mass, and still more preferably 30 parts by mass. The upper limit of the content is preferably 80 parts by mass, more preferably 70 parts by mass, and still more preferably 60 parts by mass.

### (C) Acid Diffusion Control Agent

The acid diffusion control agent (C) is an acid diffusion control agent other than the compound (Z) as the acid diffusion control agent. The acid diffusion control agent (C) is exemplified by a nitrogen atom-containing compound, a photodegradable base that is photosensitized by an exposure to generate a weak acid, and the like.

Examples of the nitrogen atom-containing compound include: amine compounds such as tripentylamine and trioctylamine; amide group-containing compounds such as formamide and N,N-dimethylacetamide; urea compounds such as urea and 1,1-dimethylurea; nitrogen-containing heterocyclic compounds such as pyridine, N-(undecylcarbonyloxyethyl) morpholine, and N-t-pentyloxycarbonyl-4-hydroxypiperidine; and the like.

Examples of the photodegradable base include a compound containing an onium cation degraded by exposure, and an anion of a weak acid; and the like. With regard to this, the cation moiety in this case does not encompass an aromatic ring structure obtained by substituting a hydrogen atom with a fluorine atom or a fluorine atom-containing group. In a light-exposed region, the photodegradable base generates a weak acid from: a proton produced upon degradation of the onium cation; and the anion of the weak acid. The anion of the weak acid preferably includes an iodine atom, and more preferably has an aromatic ring obtained by substitution with one or more iodine atoms.

Examples of the acid diffusion control agent (C) include a compound obtained by combining a substituted or unsubstituted triphenylsulfonium cation and the anion moiety (Q-2) described in the above section “(Z) Compound”, and the like.

In the case in which the radiation-sensitive resin composition contains the acid diffusion control agent (C), the lower limit of a proportion of the acid diffusion control agent (C) in the radiation-sensitive resin composition acid with respect to 100 mol % of the radiation-sensitive acid generating agent (the compound (Z) functioning as the radiation-sensitive acid generating agent and/or the acid generating agent (B)) contained in the radiation-sensitive resin composition is preferably 10 mol %, more preferably 20 mol %, and still more preferably 30 mol %. The upper limit of the proportion is preferably 90 mol %, more preferably 80 mol %, and still more preferably 70 mol %.

### (D) Organic Solvent

The radiation-sensitive resin composition typically contains the organic solvent (D). The organic solvent (D) is not particularly limited as long as it is a solvent capable of dissolving or dispersing at least the polymer (A) and the compound (Z), as well as the acid generating agent (B), the acid diffusion control agent (C), the polymer (F), and the other optional component(s) which is/are contained as needed.

The organic solvent (D) is exemplified by an alcohol solvent, an ether solvent, a ketone solvent, an amide solvent, an ester solvent, a hydrocarbon solvent, and the like. The radiation-sensitive resin composition may contain one, or two or more types of the organic solvent (D).

Examples of the alcohol solvent include:
aliphatic monohydric alcohol solvents having 1 to 18 carbon atoms such as 4-methyl-2-pentanol, n-hexanol, and diacetone alcohol;
alicyclic monohydric alcohol solvents having 3 to 18 carbon atoms such as cyclohexanol;
polyhydric alcohol solvents having 2 to 18 carbon atoms such as 1,2-propylene glycol;
polyhydric alcohol partial ether solvents having 3 to 19 carbon atoms such as propylene glycol monomethyl ether; and the like.

Examples of the ether solvent include:
dialkyl ether solvents such as diethyl ether, dipropyl ether, dibutyl ether, dipentyl ether, diisoamyl ether, dihexyl ether, and diheptyl ether;
cyclic ether solvents such as tetrahydrofuran and tetrahydropyran;
aromatic ring-containing ether solvents such as diphenyl ether and anisole; and the like.

Examples of the ketone solvent include:
chain ketone solvents such as acetone, methyl ethyl ketone, methyl n-propyl ketone, methyl n-butyl ketone, diethyl ketone, methyl iso-butyl ketone, 2-heptanone, ethyl n-butyl ketone, methyl n-hexyl ketone, di-iso-butyl ketone, and trimethylnonanone;
cyclic ketone solvents such as cyclopentanone, cyclohexanone, cycloheptanone, cyclooctanone, and methylcyclohexanone;
2,4-pentanedione, acetonylacetone, and acetophenone; and the like.

Examples of the amide solvent include:
cyclic amide solvents such as N,N′-dimethylimidazolidinone and N-methylpyrrolidone;
chain amide solvents such as N-methylformamide, N,N-dimethylformamide, N,N-diethylformamide, acetamide, N-methylacetamide, N,N-dimethylacetamide, and N-methylpropionamide; and the like.

Examples of the ester solvent include:
monocarboxylic acid ester solvents such as n-butyl acetate and ethyl lactate;
lactone solvents such as γ-butyrolactone and valerolactone;
polyhydric alcohol carboxylate solvents such as propylene glycol acetate;
polyhydric alcohol partial ether carboxylate solvents such as propylene glycol monomethyl ether acetate;
polyhydric carboxylic acid diester solvents such as diethyl oxalate;
carbonate solvents such as dimethyl carbonate and diethyl carbonate; and the like.

Examples of the hydrocarbon solvent include:
aliphatic hydrocarbon solvents having 5 to 12 carbon atoms such as n-pentane and n-hexane;
aromatic hydrocarbon solvents having 6 to 16 carbon atoms such as toluene and xylene; and the like.

The organic solvent (D) is preferably the alcohol solvent, the ester solvent or a combination of the same, more preferably the polyhydric alcohol partial ether solvent having 3 to 19 carbon atoms, the polyhydric alcohol partial ether carboxylate solvent, or a combination of the same, and still more preferably propylene glycol monomethyl ether propylene glycol monomethyl ether acetate, or a combination of the same.

In the case of the radiation-sensitive resin composition containing the organic solvent (D), the lower limit of a proportion of the organic solvent (D) with respect to total components contained in the radiation-sensitive resin composition is preferably 50% by mass, more preferably 60% by mass, still more preferably 70% by mass, and particularly preferably 80% by mass. The upper limit of the proportion is preferably 99.9% by mass, more preferably 99.5% by mass, and still more preferably 99.0% by mass.

### (F) Polymer

The polymer (F) is a polymer that differs from the polymer (A), and has a percentage content of fluorine atoms which is greater than that of the polymer (A). In general, a more hydrophobic polymer than a polymer that serves as a base polymer tends to be localized in a resist film surface layer. Since the polymer (F) has a percentage content of fluorine atoms which is greater than that of the polymer (A), due to characteristics resulting from the hydrophobicity, the polymer (F) tends to be localized in the resist film surface layer. As a result, in the case in which the radiation-sensitive resin composition contains the polymer (F), a cross-sectional shape of a resist pattern to be formed is expected to be favorable. In the case in which the radiation-sensitive resin composition contains the polymer (F), the cross-sectional shape of the resist pattern can be further improved.

The radiation-sensitive resin composition may contain the polymer (F) as, for example, a surface conditioning agent of a resist film. The radiation-sensitive resin composition may contain one type, or two or more types of the polymer (F).

The lower limit of a percentage content of fluorine atoms in the polymer (F) is preferably 1% by mass, more preferably 2% by mass, and still more preferably 3% by mass. The upper limit of the percentage content of fluorine atoms is preferably 60% by mass, more preferably 50% by mass, and still more preferably 40% by mass. It is to be noted that the percentage content by mass of fluorine atoms in the polymer may be calculated based on the structure of the polymer determined by 13 C-NMR spectroscopy.

The mode of incorporation of the fluorine atom in the polymer (F) is not particularly limited, and the fluorine atom may be bonded to either the main chain or the side chain of the polymer (F). In a preferred mode of incorporation of the fluorine atom in the polymer (F), the polymer (F) has a structural unit (hereinafter, may be also referred to as “structural unit (f)”) including a fluorine atom. The polymer (F) may further have a structural unit aside from the structural unit (F). The polymer (F) may have one, or two or more types of each structural unit.

The lower limit of the Mw of the polymer (F) as determined by GPC is preferably 2,000, more preferably 3,000, and still more preferably 5,000. The upper limit of the Mw is preferably 50,000, more preferably 20,000, and still more preferably 10,000.

The upper limit of a ratio (Mw/Mn) of the Mw to the Mn of the polymer (F) as determined by GPC is preferably 5.0, more preferably 3.0, still more preferably 2.5, and particularly preferably 2.0. The lower limit of the ratio is typically 1.0, and preferably 1.2.

In the case in which the radiation-sensitive resin composition contains the polymer (F), the lower limit of a content of the polymer (F) with respect to 100 parts by mass of the polymer (A) is preferably 0.1 parts by mass, and more preferably 0.5 parts by mass. The upper limit of the content is preferably 10 parts by mass, and more preferably 5 parts by mass.

Similarly to the polymer (A), the polymer (F) can be synthesized by, for example, polymerizing a monomer that gives each structural unit according to a well-known procedure.

Each structural unit contained in the polymer (F) is described below.

### Structural Unit (f)

The structural unit (f) is a structural that includes a fluorine atom. The percentage content of fluorine atoms in the polymer (F) can be adjusted by adjusting the proportion of the structural unit (f) in the polymer (F). Examples of the structural unit (f) include a structural unit (hereinafter, may be also referred to as “structural unit (f-1)”) represented by the following formula (f), and the like.

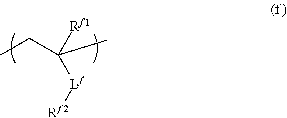

In the above formula (f), R^{f1} represents a hydrogen atom, a fluorine atom, a methyl group, or a trifluoromethyl group; L^{f} represents a single bond, an oxygen atom, a sulfur atom, —COO—, —SO₂NH—, —CONH—, or —OCONH—; and R^{f2} represents a substituted or unsubstituted monovalent fluorinated hydrocarbon group having 1 to 20 carbon atoms.

In light of copolymerizability of a monomer that gives the structural unit (f-1), R^{f1} represents preferably a hydrogen atom or a methyl group, and more preferably a methyl group.

L^{f} represents preferably —COO—.

The substituted or unsubstituted monovalent fluorinated hydrocarbon group having 1 to 20 carbon atoms represented by R is exemplified by a fluorinated alkyl group, and the like.

A part or all of hydrogen atoms in the fluorinated hydrocarbon group may be substituted with a substituent. Examples of the substituent include groups similar to those exemplified as the substituent which may be contained in the ring structure which gives Ar¹ in the above-described formula (1), and the like.

In the case in which the polymer (F) has the structural unit (f), the lower limit of a proportion of the structural unit (f) contained with respect to total structural units constituting the polymer (F) is preferably 10 mol %, more preferably 20 mol %, and still more preferably 25 mol %. The upper limit of the proportion is, for example, 100 mol %.

### Other Structural Unit(s)

Examples of the other structural unit(s) include a structural unit having an acid-labile group, and the like.

### Other Optional Component(s)

The other optional component(s) is/are exemplified by a surfactant and the like. The radiation-sensitive resin composition may contain one, or two or more types each of the other optional component(s).

### Method of Forming Resist Pattern

The method of forming a resist pattern according to the other embodiment of the present invention includes: a step (hereinafter, may be also referred to as “applying step”) of applying a radiation-sensitive resin composition directly or indirectly on a substrate; a step (hereinafter, may be also referred to as “exposing step”) of exposing a resist film formed by the applying step; and a step (hereinafter, may be also referred to as “developing step”) of developing the resist film exposed.

In the applying step, the radiation-sensitive resin composition of the one embodiment of the present invention is used as the radiation-sensitive resin composition. Thus, the method of forming a resist pattern of the other embodiment of the present invention enables forming a resist pattern being superior in CDU performance and resolution with high sensitivity.

Each step included in the method of forming a resist pattern is described below.

### Applying Step

In this step, the radiation-sensitive resin composition is applied directly or indirectly on the substrate. By this step, the resist film is formed directly or indirectly on the substrate.

In this step, the radiation-sensitive resin composition of the one embodiment of the present invention, described above, is used as the radiation-sensitive resin composition.

The substrate is exemplified by a conventionally well-known substrate such as a silicon wafer, a wafer coated with silicon dioxide or aluminum, and the like. In addition, the case of indirectly applying the radiation-sensitive resin composition on the substrate may be, for example, a case of applying the radiation-sensitive resin composition on an antireflective film formed on the substrate, and the like. Such an antireflective film is exemplified by an organic or inorganic antireflective film disclosed in, for example, Japanese Examined Patent Application, Publication No. H6-12452, Japanese Unexamined Patent Application, Publication No. S59-93448, and the like.

An application procedure is exemplified by spin coating, cast coating, roll coating, and the like. After the application, prebaking (hereinafter, may be also referred to as “PB”) may be carried out as needed for evaporating the solvent remaining in the coating film. The lower limit of a PB temperature is preferably 60° C., and more preferably 80° C. The upper limit of the PB temperature is preferably 150° C., and more preferably 140° C. The lower limit of a PB time period is preferably 5 sec, and more preferably 10 sec. The upper limit of the PB time period is preferably 600 sec, and more preferably 300 sec. The lower limit of an average thickness of the resist film formed is preferably 10 nm, and more preferably 20 nm. The upper limit of the average thickness is preferably 1,000 nm, and more preferably 500 nm.

### Exposing Step

In this step, the resist film formed by the applying step is exposed. This exposure is carried out by irradiation with an exposure light through a photomask (as the case may be, through a liquid immersion medium such as water). Examples of the exposure light include: electromagnetic waves such as visible light rays, ultraviolet rays, far ultraviolet rays, extreme ultraviolet rays (EUV), X-rays, and γ-rays; charged particle rays such as electron beams and α-rays; and the like, which may be selected in accordance with a line width of the intended pattern, and the like. Of these, far ultraviolet rays, EUV, or electron beams are preferred; an ArF excimer laser beam (wavelength: 193 nm), a KrF excimer laser beam (wavelength: 248 nm), EUV (wavelength: 13.5 nm), or electron beams are more preferred; a KrF excimer laser beam, EUV, or electron beams are still more preferred; and EUV or electron beams are particularly preferred.

It is preferred that post exposure baking (hereinafter, may be also referred to as “PEB”) is carried out after the exposure to promote dissociation of the acid-labile group included in the polymer (A) etc., mediated by the acid generated from the compound (Z), the acid generating agent (B), etc., upon the exposure in exposed regions of the resist film. This

PEB enables an increase in a difference in solubility of the resist film in a developer solution between the light-exposed regions and light-unexposed regions. The lower limit of a temperature of the PEB is preferably 50° C., and more preferably 80° C. The upper limit of the temperature of the PEB is preferably 180° C., and more preferably 130° C. The lower limit of a time period of the PEB is preferably 5 sec, more preferably 10 sec, and still more preferably 30 sec. The upper limit of the time period of the PEB is preferably 600 sec, more preferably 300 sec, and still more preferably 100 sec.

### Developing Step

In this step, the resist film exposed is developed. Accordingly, formation of a predetermined resist pattern is enabled. After the development, washing with a rinse agent such as water or an alcohol and then drying is typically performed. The development procedure in the developing step may be carried out by either development with an alkali, or development with an organic solvent.

In the case of the development with an alkali, the developer solution for use in the development is exemplified by alkaline aqueous solutions prepared by dissolving at least one alkaline compound such as sodium hydroxide, potassium hydroxide, sodium carbonate, sodium silicate, sodium metasilicate, aqueous ammonia, ethylamine, n-propylamine, diethylamine, di-n-propylamine, triethylamine, methyldiethylamine, ethyldimethylamine, triethanolamine, tetramethylammonium hydroxide (hereinafter, may be also referred to as “TMAH”), pyrrole, piperidine, choline, 1,8-diazabicyclo-[5.4.0]-7-undecene, and 1,5-diazabicyclo-[4.3.0]-5-nonene; and the like. Of these, an aqueous TMAH solution is preferred, and a 2.38% by mass aqueous TMAH solution is more preferred.

In the case of the development with an organic solvent, the developer solution is exemplified by: an organic solvent such as a hydrocarbon solvent, an ether solvent, an ester solvent, a ketone solvent, and an alcohol solvent; a solution containing the organic solvent; and the like. Exemplary organic solvents include the solvents exemplified as the organic solvent (D) for the radiation-sensitive resin composition, and the like. Of these, the ester solvent or the ketone solvent is preferred. The ester solvent is preferably an acetic acid ester solvent, and more preferably n-butyl acetate. The ketone solvent is preferably a chain ketone, and more preferably 2-heptanone. The lower limit of the content of the organic solvent in the developer solution is preferably 80% by mass, more preferably 90% by mass, still more preferably 95% by mass, and particularly preferably 99% by mass. Components other than the organic solvent in the organic solvent developer solution are exemplified by water, silicone oil, and the like.

Examples of the development procedure include: a dipping procedure in which the substrate is immersed for a given time period in the developer solution charged in a container; a puddle procedure in which the developer solution is placed to form a dome-shaped bead by way of the surface tension on the surface of the substrate for a given time period to conduct a development; a spraying procedure in which the developer solution is sprayed onto the surface of the substrate; a dynamic dispensing procedure in which the developer solution is continuously discharged onto the substrate, which is rotated at a constant speed, while scanning with a developer solution-discharging nozzle at a constant speed; and the like.

The resist pattern to be formed according to the method of forming a resist pattern is exemplified by a line-and-space pattern, a contact hole pattern, and the like.

### EXAMPLES

Hereinafter, the present invention is explained in detail by way of Examples, but the present invention is not in any way limited to these Examples. Measuring methods for various types of physical property values are shown below.

Weight Average Molecular Weight (Mw), Number Average Molecular Weight (Mn), and Polydispersity Index (Mw/Mn)

Measurements of the Mw and the Mn of the polymer were carried out in accordance with the conditions described in the aforementioned paragraph “Method for Measuring Mw and Mn”. The polydispersity index (Mw/Mn) of the polymer was calculated from the measurement results of the Mw and the Mn.

### Synthesis of Monomer (X)

Compounds (hereinafter, may be also referred to as “monomers (X-1) to (X-14)”) represented by the following formulae (X-1) to (X-14) as the monomer (X) were synthesized in accordance with the following method

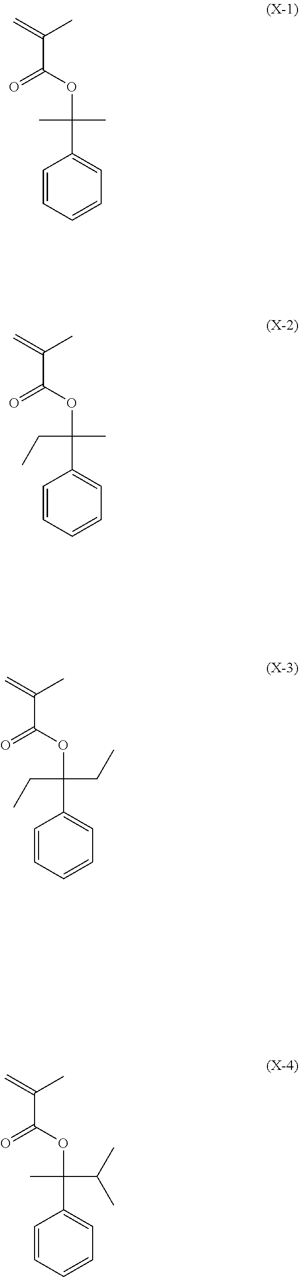

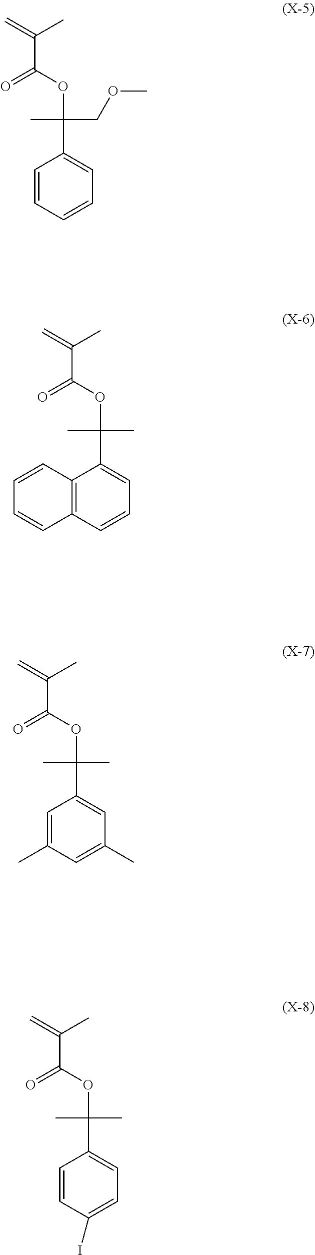

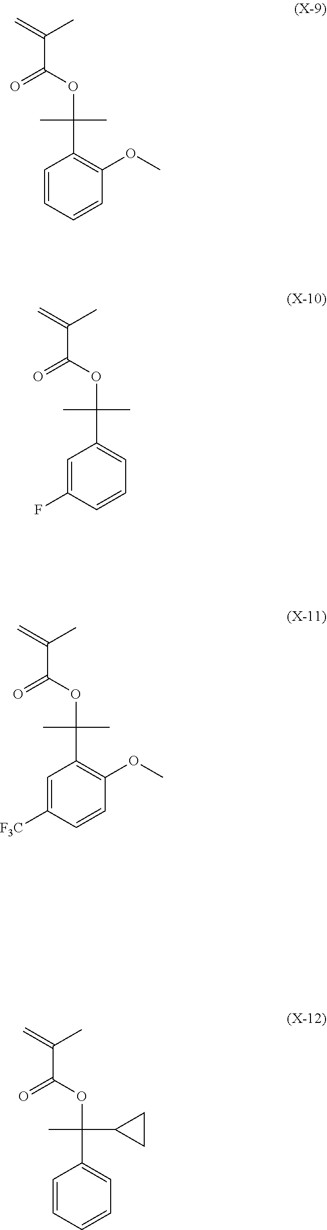

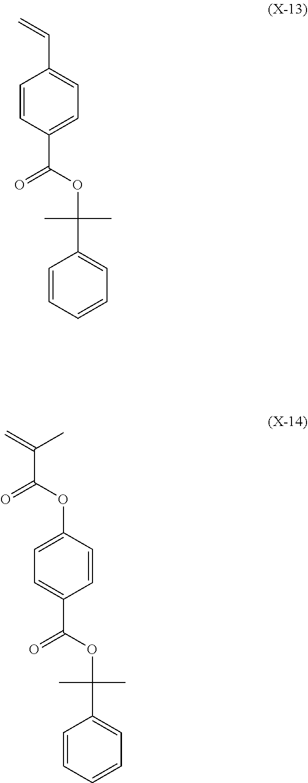 [SMILES: C=Cc1ccc(C(=O)OC(C)(C)c2ccccc2)cc1 ; C=C(C)C(=O)Oc1ccc(C(=O)OC(C)(C)c2ccccc2)cc1]

### Synthesis Example 1-1: Synthesis of Monomer (X-1)

Into a vessel containing tetrahydrofuran (300 mL) was charged a compound (hereinafter, may be also referred to as “compound (P-1)”) (150 mmol) represented by the following formulae (P-1), and a thus resulting mixture was cooled to 0° C. Into this vessel, 180 mL of a 1 mol/L solution of methylmagnesium bromide in tetrahydrofuran was added dropwise. Stirring was conducted for 8 hrs at room temperature. After cooling to 0° C., an aqueous solution of ammonium chloride, and ethyl acetate were charged thereto. An organic layer was washed with a saline solution, and then ultra-pure water, in this order. The organic layer was dried over sodium sulfate and then filtered off. The solution was distilled to give a compound (hereinafter, may be also referred to as “compound P-2)”) represented by the following formula (P-2).

Into a vessel containing acetonitrile (150 mL) were charged the compound (P-2) (150 mmol) and triethylamine (180 mmol), and a thus resulting mixture was cooled to 0° C. Into this vessel, chloride methacrylate (180 mmol) was added dropwise. Stirring was conducted for 3 hrs at room temperature, and then an aqueous solution of ammonium chloride, and ethyl acetate were charged thereto. An organic layer was washed with a saline solution, and then ultra-pure water, in this order. The organic layer was dried over sodium sulfate and then filtered off. The solution was distilled to give a monomer (X-1).

A synthesis scheme of the monomer (X-1) is shown below. In the following synthesis scheme, MeMgBr represents methylmagnesium bromide, and NEt₃ represents triethylamine.

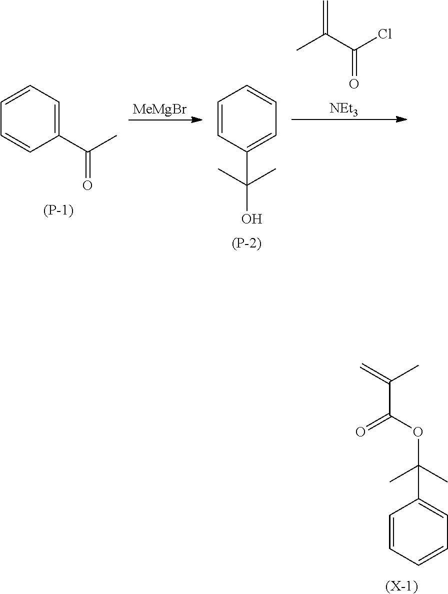

### Synthesis Examples 1-2 to 1-14: Syntheses of Monomers (X-2) to (X-14)

Monomers (X-2) to (X-14) were synthesized similarly to Synthesis Example 1-1, except for appropriately selecting each precursor.

### Synthesis of Polymer (A)

Each of monomers (A-1) to (A-29), (A-31) to (A-35), and (CA-1) as the polymer (A) was synthesized in accordance with the following method. For syntheses of the polymers (A), the monomers (X-1) to (X-14), and compounds (hereinafter, may be also referred to as “monomers (M-1) to (M-19)”) represented by the following formulae (M-1) to (M-19) were used. In the following Synthesis Examples, unless otherwise specified particularly, the term “parts by mass” means a value, provided that the total mass of the monomers used was 100 parts by mass, and the term “mol %” means a value, provided that the total mol number of the monomers used was 100 mol %. In the following structural formulae, “(F-Ph)₃S⁺” represents a tris(4-fluorophenyl) sulfonium cation.

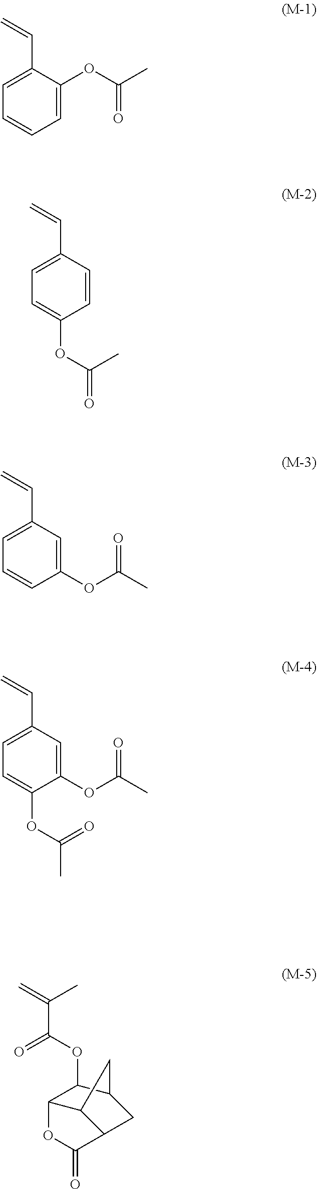

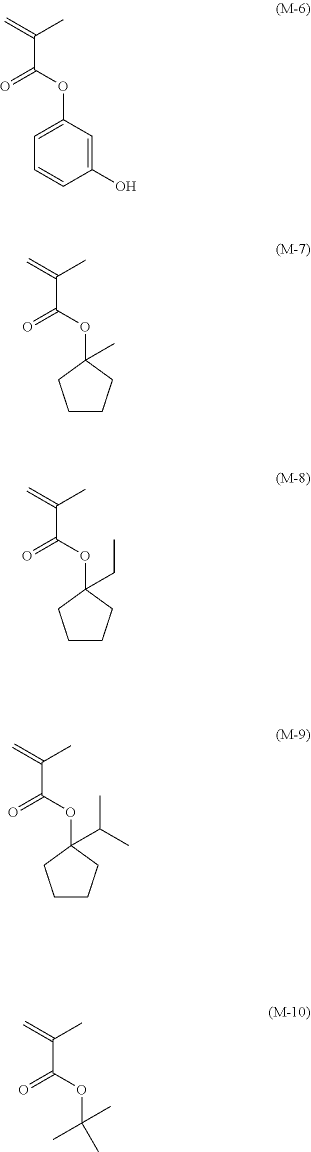

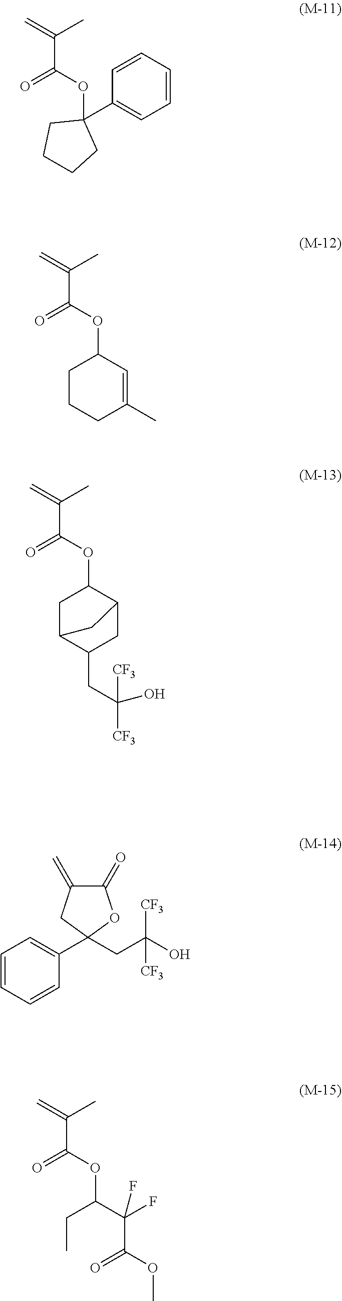

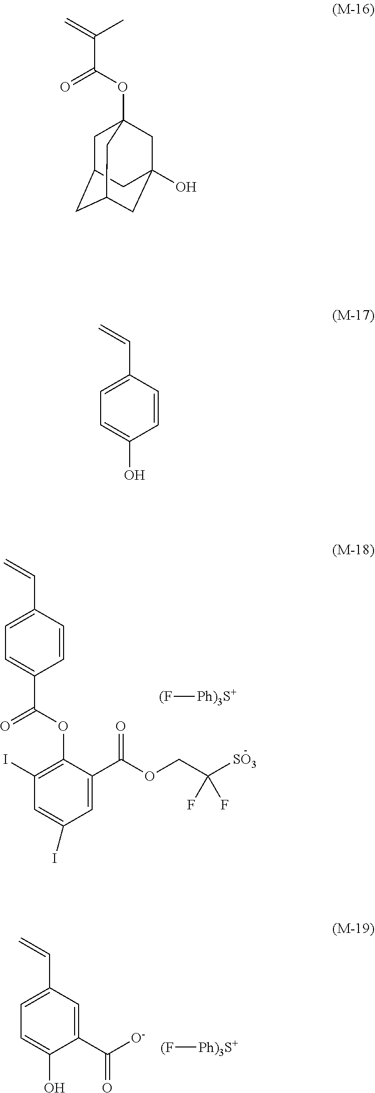

### Synthesis Example 2-1: Synthesis of Polymer (A-1)

The monomer (X-1), the monomer (M-2), and the monomer (M-8) were dissolved in propylene glycol monomethyl ether (200 parts by mass) such that a molar ratio of the monomers became 20/45/35. Thereto was added as an initiator, 2,2′-azobis(methylisobutyrate) (10 mol %) to prepare a monomer solution. Meanwhile, propylene glycol monomethyl ether (100 parts by mass with respect to a total amount of the monomers) was charged into an empty reaction vessel, and heated to 85° C. with stirring. Into this vessel, the monomer solution was added dropwise over 3 hours. After completion of the dropwise addition, the mixture was heated at 85° C. for an additional 3 hours, whereby the polymerization reaction was performed for a total of 6 hrs. After completion of the polymerization reaction, the polymerization solution was cooled to room temperature. The polymerization solution was added dropwise into n-hexane (1,000 parts by mass) to allow for solidification purification of the polymer. To the polymer thus collected were added again propylene glycol monomethyl ether (150 parts by mass), methanol (150 parts by mass), triethylamine (1.5 molar equivalent with respect to the usage amount of the compound (M-2)), and water (1.5 molar equivalent with respect to the usage amount of the compound (M-2)). Reflux was allowed at a boiling point to carry out a hydrolysis reaction for 8 hrs. After completion of the reaction, the solvent and triethylamine were distilled off under reduced pressure, and a thus obtained polymer was dissolved in acetone (150 parts by mass). This solution was added dropwise into water (2,000 parts by mass) to permit coagulation, and thus produced white powder was filtered. The white powder was dried at 50° C. for 17 hours to give a white powdery polymer (A-1) with a favorable yield. With respect to the polymer (A-1), the Mw was 5,800, and the Mw/Mn was 1.5.

### Synthesis Examples 2-2 to 2-33: Syntheses of Polymers (A-2) to (A-29), (A-31) to (A-33), and (CA-1)

Polymers (A-2) to (A-29), (A-31) to (A-33), and (CA-1) were synthesized similarly to Synthesis Example 2-1, except that each monomer of the type and in a blend proportion as shown in Table 1 below was used.

### Synthesis Examples 2-34 to 2-35: Syntheses of Polymers (A-34) to (A-35)

Each monomer was combined and copolymerization thereof was allowed to proceed in the presence of a tetrahydrofuran (THF) solvent, followed by isolation and drying, to give the polymers (A-34) to (A-35), each having a formulation as shown below. The formulation of the each of the polymers obtained was confirmed by ¹H-NMR, and the Mw and dispersity index (Mw/Mn) thereof was confirmed in accordance with the above-described GPC conditions.

Types and usage proportions of the monomers that give each structural unit in the polymers obtained in Synthesis Examples 2-1 to 2-35, as well as the Mw and the Mw/Mn thereof, are shown in Table 1 below. It is to be noted that in Table 1 below, “-” indicates that the corresponding monomer was not used.

TABLE 1

| | | Monomer that gives | | Monomer that gives | |
|---|---|---|---|---|---|
| | | structural unit (I) | | structural unit (II) | |
| | | | usage amount | | usage amount |
| | (A) Polymer | type | (% by mole) | type | (% by mole) |
| Synthesis Example 2-1 | A-1 | X-1 | 20 | M-2 | 45 |
| Synthesis Example 2-2 | A-2 | X-2 | 20 | M-2 | 45 |
| Synthesis Example 2-3 | A-3 | X-3 | 20 | M-2 | 45 |
| Synthesis Example 2-4 | A-4 | X-4 | 20 | M-2 | 45 |
| Synthesis Example 2-5 | A-5 | X-5 | 20 | M-2 | 45 |
| Synthesis Example 2-6 | A-6 | X-6 | 20 | M-2 | 45 |
| Synthesis Example 2-7 | A-7 | X-7 | 20 | M-2 | 45 |
| Synthesis Example 2-8 | A-8 | X-8 | 20 | M-2 | 45 |
| Synthesis Example 2-9 | A-9 | X-9 | 20 | M-2 | 45 |
| Synthesis Example 2-10 | A-10 | X-10 | 20 | M-2 | 45 |
| Synthesis Example 2-11 | A-11 | X-11 | 20 | M-2 | 45 |
| Synthesis Example 2-12 | A-12 | X-12 | 20 | M-2 | 45 |
| Synthesis Example 2-13 | A-13 | X-1 | 55 | M-2 | 45 |
| Synthesis Example 2-14 | A-14 | X-2 | 55 | M-2 | 45 |
| Synthesis Example 2-15 | A-15 | X-6 | 55 | M-2 | 45 |
| Synthesis Example 2-16 | A-16 | X-8 | 55 | M-2 | 45 |
| Synthesis Example 2-17 | A-17 | X-10 | 55 | M-2 | 45 |
| Synthesis Example 2-18 | A-18 | X-1 | 20 | M-2 | 45 |
| Synthesis Example 2-19 | A-19 | X-1 | 20 | M-2 | 45 |
| Synthesis Example 2-20 | A-20 | X-1 | 20 | M-2 | 45 |
| Synthesis Example 2-21 | A-21 | X-1 | 20 | M-2 | 45 |
| Synthesis Example 2-22 | A-22 | X-1 | 20 | M-2 | 45 |
| Synthesis Example 2-23 | A-23 | X-1 | 20 | M-2/M-3 | 25/20 |
| Synthesis Example 2-24 | A-24 | X-1 | 20 | M-2/M-1 | 25/20 |
| Synthesis Example 2-25 | A-25 | X-1 | 20 | M-2/M-4 | 25/20 |
| Synthesis Example 2-26 | A-26 | X-1 | 20 | M-2 | 25 |
| Synthesis Example 2-27 | A-27 | X-1 | 20 | M-2/M-6 | 25/20 |
| Synthesis Example 2-28 | A-28 | X-1 | 20 | M-2 | 25 |
| Synthesis Example 2-29 | A-29 | X-1 | 20 | M-2 | 25 |
| Synthesis Example 2-30 | CA-1 | — | — | M-2 | 45 |
| Synthesis Example 2-31 | A-31 | X-13 | 20 | M-2 | 45 |
| Synthesis Example 2-32 | A-32 | X-14 | 20 | M-2 | 45 |
| Synthesis Example 2-33 | A-33 | X-1 | 20 | M-2 | 25 |
| Synthesis Example 2-34 | A-34 | X-1 | 20 | M-17 | 37 |
| Synthesis Example 2-35 | A-35 | X-1 | 20 | M-17 | 40 |

| | | | Monomer that | | | |
|---|---|---|---|---|---|---|
| | Monomer that gives | | gives other | | | |
| | structural unit (III) | | structural unit(s) | | | |
| | | usage amount | | usage amount | | |
| | type | (% by mole) | type | (% by mole) | Mw | Mw/Mn |
| Synthesis Example 2-1 | M-8 | 35 | — | — | 5,800 | 1.5 |
| Synthesis Example 2-2 | M-8 | 35 | — | — | 5,900 | 1.5 |
| Synthesis Example 2-3 | M-8 | 35 | — | — | 6,300 | 1.5 |
| Synthesis Example 2-4 | M-8 | 35 | — | — | 6,400 | 1.6 |
| Synthesis Example 2-5 | M-8 | 35 | — | — | 6,200 | 1.5 |
| Synthesis Example 2-6 | M-8 | 35 | — | — | 6,400 | 1.5 |
| Synthesis Example 2-7 | M-8 | 35 | — | — | 5,300 | 1.5 |
| Synthesis Example 2-8 | M-8 | 35 | — | — | 4,000 | 1.6 |
| Synthesis Example 2-9 | M-8 | 35 | — | — | 4,900 | 1.6 |
| Synthesis Example 2-10 | M-8 | 35 | — | — | 5,700 | 1.5 |
| Synthesis Example 2-11 | M-8 | 35 | — | — | 5,800 | 1.5 |
| Synthesis Example 2-12 | M-8 | 35 | — | — | 6,100 | 1.4 |
| Synthesis Example 2-13 | — | — | — | — | 6,200 | 1.5 |
| Synthesis Example 2-14 | — | — | — | — | 6,300 | 1.4 |
| Synthesis Example 2-15 | — | — | — | — | 6,000 | 1.5 |
| Synthesis Example 2-16 | — | — | — | — | 5,500 | 1.5 |
| Synthesis Example 2-17 | — | — | — | — | 5,800 | 1.4 |
| Synthesis Example 2-18 | M-7 | 35 | — | — | 5,700 | 1.5 |
| Synthesis Example 2-19 | M-9 | 35 | — | — | 5,900 | 1.6 |
| Synthesis Example 2-20 | M-10 | 35 | — | — | 6,100 | 1.6 |
| Synthesis Example 2-21 | M-11 | 35 | — | — | 6,200 | 1.5 |
| Synthesis Example 2-22 | M-12 | 35 | — | — | 5,100 | 1.6 |
| Synthesis Example 2-23 | M-8 | 35 | — | — | 5,400 | 1.6 |
| Synthesis Example 2-24 | M-8 | 35 | — | — | 5,300 | 1.6 |
| Synthesis Example 2-25 | M-8 | 35 | — | — | 6,000 | 1.5 |
| Synthesis Example 2-26 | M-8 | 35 | M-5 | 20 | 6,700 | 1.5 |
| Synthesis Example 2-27 | M-8 | 35 | — | — | 5,900 | 1.5 |
| Synthesis Example 2-28 | M-8 | 35 | M-13 | 20 | 5,900 | 1.4 |
| Synthesis Example 2-29 | M-8 | 35 | M-14 | 20 | 6,700 | 1.6 |
| Synthesis Example 2-30 | M-8 | 55 | — | — | 4,900 | 1.5 |
| Synthesis Example 2-31 | M-8 | 35 | — | — | 5,700 | 1.5 |
| Synthesis Example 2-32 | M-8 | 35 | — | — | 5,200 | 1.5 |
| Synthesis Example 2-33 | M-8 | 35 | M-16 | 20 | 6,500 | 1.6 |
| Synthesis Example 2-34 | M-8 | 35 | M-18 | 8 | 7,600 | 1.5 |
| Synthesis Example 2-35 | M-8 | 35 | M-19 | 5 | 7,700 | 1.5 |

### Synthesis of Polymer (F)

Polymer (F-1) as the polymer (F) was synthesized in accordance with the following method. For synthesis of the polymer (F), the monomer (M-9) and the monomer (M-15) were used.

### Synthesis Example 3-1: Synthesis of Polymer (F-1)

The monomer (M-9) and the monomer (M-15) were dissolved in 2-butanone (100 parts by mass with respect to a total monomer amount) such that a molar ratio of the monomers became 40/60. Thereto was added as an initiator, azobisisobutyronitrile (5 mol % with respect to total monomers) to prepare a monomer solution. Meanwhile, 2-butanone (50 parts by mass) was charged into an empty vessel, and nitrogen was purged for 30 min. The interior of this vessel was heated to 80° C., and the monomer solution was added dropwise over 3 hrs with stirring. After completion of the dropwise addition, the mixture was heated at 80° C. for an additional 3 hours, and then the polymerization solution was cooled to 30° C. or below. After the polymerization solution was transferred into a separatory funnel, hexane (150 parts by mass) was added to uniformly dilute the polymerization solution. Furthermore, methanol (600 parts by mass) and water (30 parts by mass) were charged and mixed. After the mixture was left to stand for 30 min, the underlayer was recovered and the solvent was replaced with propylene glycol monomethyl ether acetate to give a 10% by mass solution of the polymer (F-1) in propylene glycol monomethyl ether acetate. With respect to the polymer (F-1), the Mw was 7,200, and the Mw/Mn was 1.7.

### Preparation of Radiation-Sensitive Resin Composition

The acid generating agent (B), the acid diffusion control agent (C), and the organic solvent (D) used in preparation of the radiation-sensitive resin composition are shown below. In the following Examples and Comparative Examples, unless otherwise specified particularly, the term “parts by mass” means a value, provided that the mass of the polymer (A) used was 100 parts by mass, and the term “mol %” means a value, provided that the mol number of the acid generating agent (B) used was 100 mol %.

### (B) Acid Generating Agent

Compounds (hereinafter, may be also referred to as “acid generating agents (B-1) to (B-13) and (CB-1) to (CB-3)”) represented by the following formulae (B-1) to (B-13) and (CB-1) to (CB-3) were used as the acid generating agent (B). The acid generating agents (B-1) to (B-13) correspond to the compound (Z).

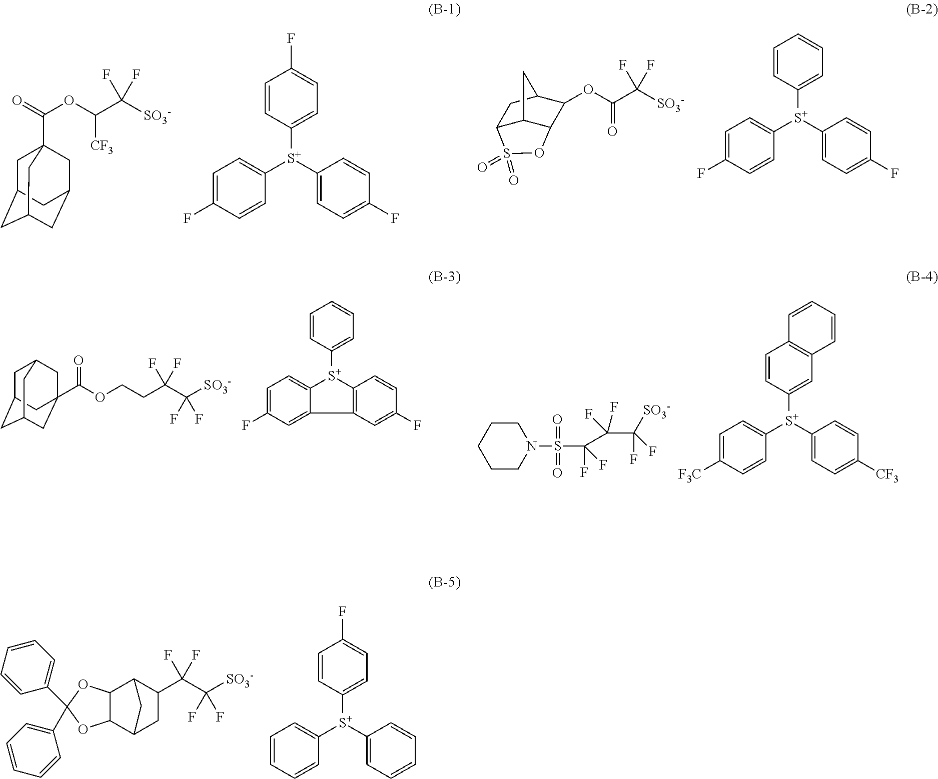

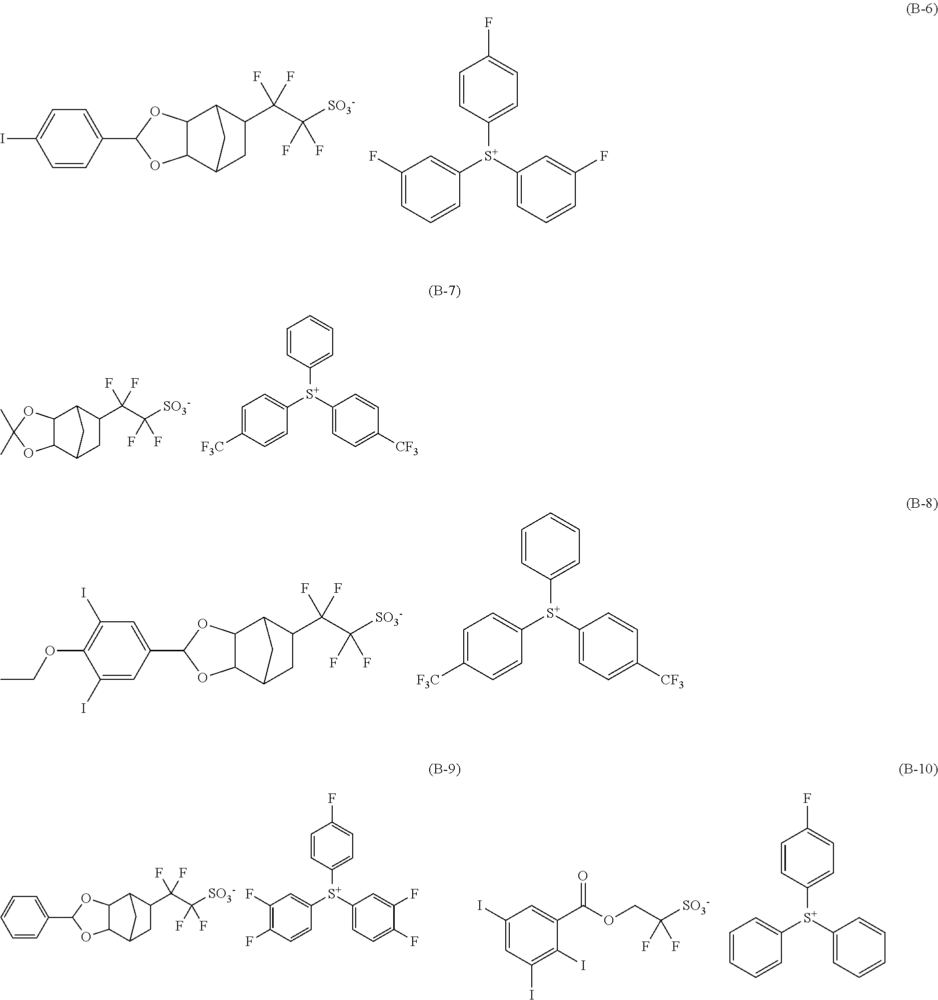

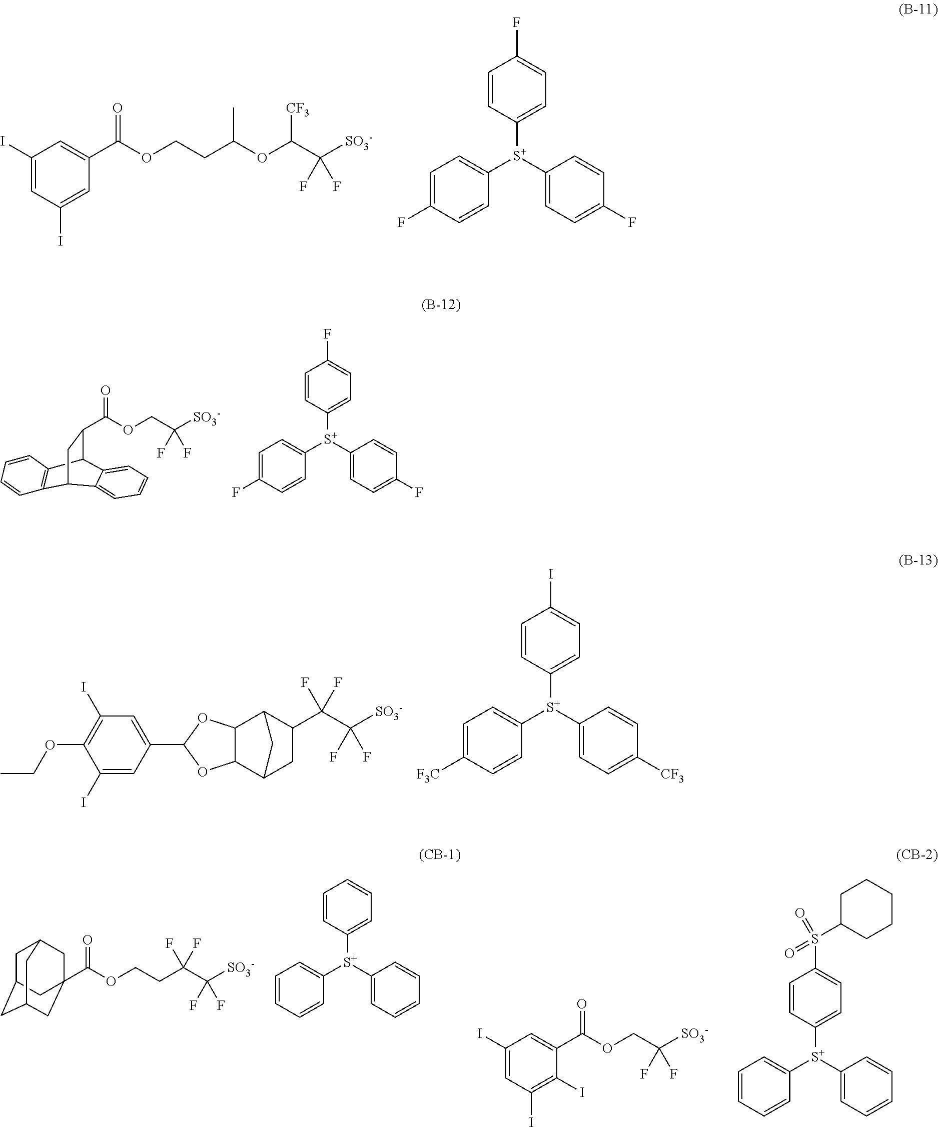

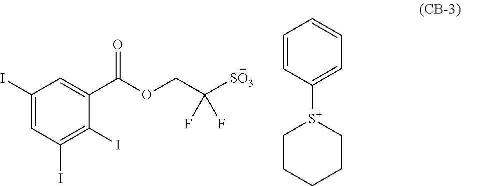

### (C) Acid Diffusion Control Agent

Compounds (hereinafter, may be also referred to as “acid diffusion control agents (C-1) to (C-9) and (CC-1)”) represented by the following formulae (C-1) to (C-9) and (CC-1) were used as the acid diffusion control agent (C). The acid diffusion control agents (C-1) to (C-9) correspond to the compound (Z).

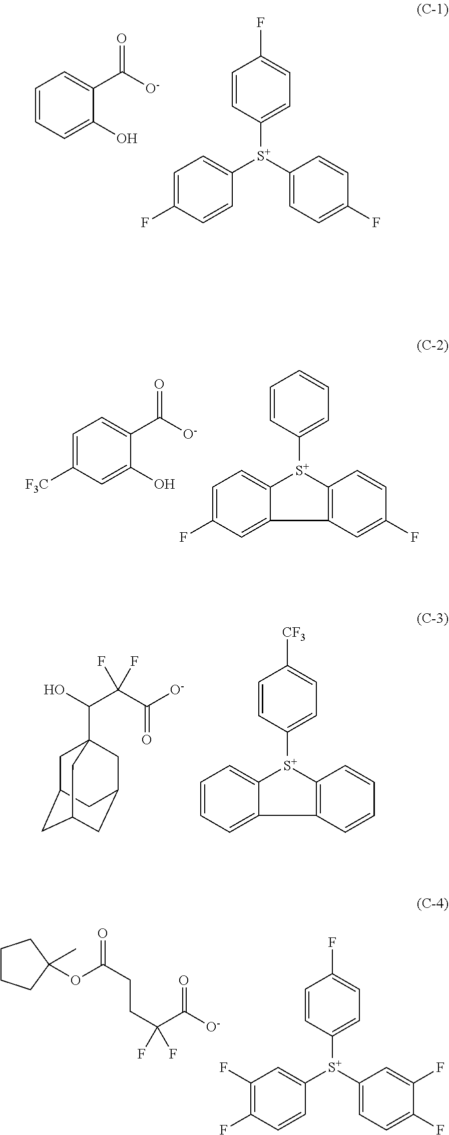

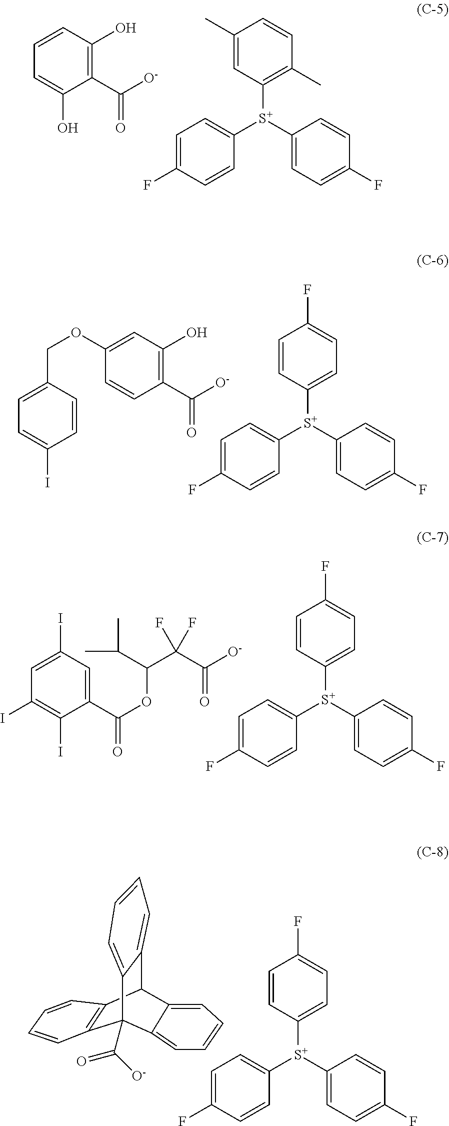

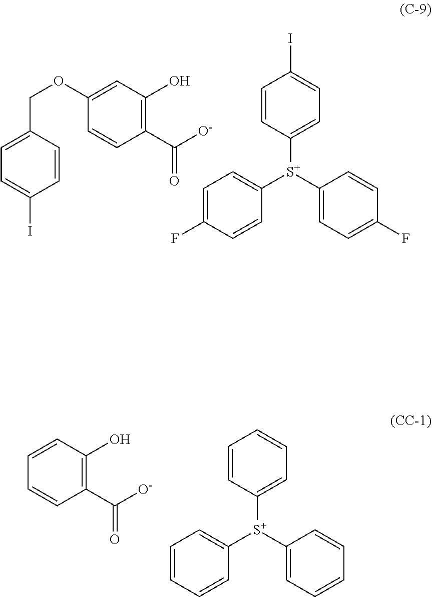 [SMILES: O=C([O-])c1ccc(OCc2ccc(I)cc2)cc1O ; Fc1ccc([S+](c2ccc(F)cc2)c2ccc(I)cc2)cc1 ; O=C([O-])c1ccccc1O ; c1ccc([S+](c2ccccc2)c2ccccc2)cc1]

### (D) Organic Solvent

The following organic solvents were used as the organic solvent (D).
(D-1): propylene glycol monomethyl ether acetate
(D-2): propylene glycol monomethyl ether

### Example 1: Preparation of Radiation-Sensitive Resin Composition (R-1)

100 parts by mass of (A-1) as the polymer (A), 45 parts by mass of (B-1) as the acid generating agent (B), (C-1) as the acid diffusion control agent (C) in an amount of 50 mol % with respect to (B-1), 5,500 parts by mass of (D-1) and 1,500 parts by mass of (D-2) as the organic solvent (D), and 3 parts by mass in terms of solid content of (F-1) as the polymer (F) were admixed. A mix liquid thus obtained was filtered through a filter having a pore size of 0.2 μm, whereby a radiation-sensitive resin composition (R-1) was prepared.

### Examples 2 to 59 and Comparative Examples 1 to 3: Preparation of Radiation-Sensitive Resin Compositions (R-2) to (R-59) and (CR-1) to (CR-3)

Radiation-sensitive resin compositions (R-2) to (R-59) and (CR-1) to (CR-3) were prepared similarly to Example 1, except that each component of the following type and in the following content shown in Table 2 below was used.

TABLE 2

| | Radiation- | (A) Polymer | | (F) Polymer | | (B) Acid generating agent | | (C) Acid diffusion | | | |
|---|---|---|---|---|---|---|---|---|---|---|---|
| | sensitive | | content | | content | | content | control agent | | (D) Solvent | |
| | resin | | (parts by | | (parts by | | (parts by | | content (% | | content (parts by |
| | composition | type | mass) | type | mass) | type | mass) | type | by mole) | type | mass) |
| Example 1 | R-1 | A-1 | 100 | F-1 | 3 | B-1 | 45 | C-1 | 50 | D-1/D-2 | 5,500/1,500 |
| Example 2 | R-2 | A-2 | 100 | F-1 | 3 | B-1 | 45 | C-1 | 50 | D-1/D-2 | 5,500/1,500 |
| Example 3 | R-3 | A-3 | 100 | F-1 | 3 | B-1 | 45 | C-1 | 50 | D-1/D-2 | 5,500/1,500 |
| Example 4 | R-4 | A-4 | 100 | F-1 | 3 | B-1 | 45 | C-1 | 50 | D-1/D-2 | 5,500/1,500 |
| Example 5 | R-5 | A-5 | 100 | F-1 | 3 | B-1 | 45 | C-1 | 50 | D-1/D-2 | 5,500/1,500 |
| Example 6 | R-6 | A-6 | 100 | F-1 | 3 | B-1 | 45 | C-1 | 50 | D-1/D-2 | 5,500/1,500 |
| Example 7 | R-7 | A-7 | 100 | F-1 | 3 | B-1 | 45 | C-1 | 50 | D-1/D-2 | 5,500/1,500 |
| Example 8 | R-8 | A-8 | 100 | F-1 | 3 | B-1 | 45 | C-1 | 50 | D-1/D-2 | 5,500/1,500 |
| Example 9 | R-9 | A-9 | 100 | F-1 | 3 | B-1 | 45 | C-1 | 50 | D-1/D-2 | 5,500/1,500 |
| Example 10 | R-10 | A-10 | 100 | F-1 | 3 | B-1 | 45 | C-1 | 50 | D-1/D-2 | 5,500/1,500 |
| Example 11 | R-11 | A-11 | 100 | F-1 | 3 | B-1 | 45 | C-1 | 50 | D-1/D-2 | 5,500/1,500 |
| Example 12 | R-12 | A-12 | 100 | F-1 | 3 | B-1 | 45 | C-1 | 50 | D-1/D-2 | 5,500/1,500 |
| Example 13 | R-13 | A-13 | 100 | F-1 | 3 | B-1 | 45 | C-1 | 50 | D-1/D-2 | 5,500/1,500 |
| Example 14 | R-14 | A-14 | 100 | F-1 | 3 | B-1 | 45 | C-1 | 50 | D-1/D-2 | 5,500/1,500 |
| Example 15 | R-15 | A-15 | 100 | F-1 | 3 | B-1 | 45 | C-1 | 50 | D-1/D-2 | 5,500/1,500 |
| Example 16 | R-16 | A-16 | 100 | F-1 | 3 | B-1 | 45 | C-1 | 50 | D-1/D-2 | 5,500/1,500 |
| Example 17 | R-17 | A-17 | 100 | F-1 | 3 | B-1 | 45 | C-1 | 50 | D-1/D-2 | 5,500/1,500 |
| Example 18 | R-18 | A-18 | 100 | F-1 | 3 | B-1 | 45 | C-1 | 50 | D-1/D-2 | 5,500/1,500 |
| Example 19 | R-19 | A-19 | 100 | F-1 | 3 | B-1 | 45 | C-1 | 50 | D-1/D-2 | 5,500/1,500 |
| Example 20 | R-20 | A-20 | 100 | F-1 | 3 | B-1 | 45 | C-1 | 50 | D-1/D-2 | 5,500/1,500 |
| Example 21 | R-21 | A-21 | 100 | F-1 | 3 | B-1 | 45 | C-1 | 50 | D-1/D-2 | 5,500/1,500 |
| Example 22 | R-22 | A-22 | 100 | F-1 | 3 | B-1 | 45 | C-1 | 50 | D-1/D-2 | 5,500/1,500 |
| Example 23 | R-23 | A-23 | 100 | F-1 | 3 | B-1 | 45 | C-1 | 50 | D-1/D-2 | 5,500/1,500 |
| Example 24 | R-24 | A-24 | 100 | F-1 | 3 | B-1 | 45 | C-1 | 50 | D-1/D-2 | 5,500/1,500 |
| Example 25 | R-25 | A-25 | 100 | F-1 | 3 | B-1 | 45 | C-1 | 50 | D-1/D-2 | 5,500/1,500 |
| Example 26 | R-26 | A-26 | 100 | F-1 | 3 | B-1 | 45 | C-1 | 50 | D-1/D-2 | 5,500/1,500 |
| Example 27 | R-27 | A-27 | 100 | F-1 | 3 | B-1 | 45 | C-1 | 50 | D-1/D-2 | 5,500/1,500 |
| Example 28 | R-28 | A-28 | 100 | F-1 | 3 | B-1 | 45 | C-1 | 50 | D-1/D-2 | 5,500/1,500 |
| Example 29 | R-29 | A-29 | 100 | F-1 | 3 | B-1 | 45 | C-1 | 50 | D-1/D-2 | 5,500/1,500 |
| Example 30 | R-30 | A-1 | 100 | F-1 | 3 | B-2 | 45 | C-1 | 50 | D-1/D-2 | 5,500/1,500 |
| Example 31 | R-31 | A-1 | 100 | F-1 | 3 | B-3 | 45 | C-1 | 50 | D-1/D-2 | 5,500/1,500 |
| Example 32 | R-32 | A-1 | 100 | F-1 | 3 | B-4 | 45 | C-1 | 50 | D-1/D-2 | 5,500/1,500 |
| Example 33 | R-33 | A-1 | 100 | F-1 | 3 | B-5 | 45 | C-1 | 50 | D-1/D-2 | 5,500/1,500 |
| Example 34 | R-34 | A-1 | 100 | F-1 | 3 | B-6 | 45 | C-1 | 50 | D-1/D-2 | 5,500/1,500 |
| Example 35 | R-35 | A-1 | 100 | F-1 | 3 | B-7 | 45 | C-1 | 50 | D-1/D-2 | 5,500/1,500 |
| Example 36 | R-36 | A-1 | 100 | F-1 | 3 | B-8 | 45 | C-1 | 50 | D-1/D-2 | 5,500/1,500 |
| Example 37 | R-37 | A-1 | 100 | F-1 | 3 | B-9 | 45 | C-1 | 50 | D-1/D-2 | 5,500/1,500 |
| Example 38 | R-38 | A-1 | 100 | F-1 | 3 | B-10 | 45 | C-1 | 50 | D-1/D-2 | 5,500/1,500 |
| Example 39 | R-39 | A-1 | 100 | F-1 | 3 | B-11 | 45 | C-1 | 50 | D-1/D-2 | 5,500/1,500 |
| Example 40 | R-40 | A-1 | 100 | F-1 | 3 | B-1 | 45 | C-2 | 50 | D-1/D-2 | 5,500/1,500 |
| Example 41 | R-41 | A-1 | 100 | F-1 | 3 | B-1 | 45 | C-3 | 50 | D-1/D-2 | 5,500/1,500 |
| Example 42 | R-42 | A-1 | 100 | F-1 | 3 | B-1 | 45 | C-4 | 50 | D-1/D-2 | 5,500/1,500 |
| Example 43 | R-43 | A-1 | 100 | F-1 | 3 | B-1 | 45 | C-5 | 50 | D-1/D-2 | 5,500/1,500 |
| Example 44 | R-44 | A-1 | 100 | F-1 | 3 | B-1 | 45 | C-6 | 50 | D-1/D-2 | 5,500/1,500 |
| Example 45 | R-45 | A-1 | 100 | F-1 | 3 | B-1 | 45 | C-7 | 50 | D-1/D-2 | 5,500/1,500 |
| Example 46 | R-46 | A-1 | 100 | F-1 | 3 | CB-1 | 45 | C-1 | 50 | D-1/D-2 | 5,500/1,500 |
| Example 47 | R-47 | A-1 | 100 | F-1 | 3 | B-1 | 45 | CC-1 | 50 | D-1/D-2 | 5,500/1,500 |
| Example 48 | R-48 | A-1 | 100 | — | — | B-1 | 45 | C-1 | 50 | D-1/D-2 | 5,500/1,500 |
| Example 49 | R-49 | A-31 | 100 | F-1 | 3 | B-1 | 45 | C-1 | 50 | D-1/D-2 | 5,500/1,500 |
| Example 50 | R-50 | A-32 | 100 | F-1 | 3 | B-1 | 45 | C-1 | 50 | D-1/D-2 | 5,500/1,500 |
| Example 51 | R-51 | A-33 | 100 | F-1 | 3 | B-1 | 45 | C-1 | 50 | D-1/D-2 | 5,500/1,500 |
| Example 52 | R-52 | A-34 | 100 | F-1 | 3 | — | — | C-1 | 50 | D-1/D-2 | 5,500/1,500 |
| Example 53 | R-53 | A-35 | 100 | F-1 | 3 | B-1 | 45 | — | — | D-1/D-2 | 5,500/1,500 |
| Example 54 | R-54 | A-1 | 100 | F-1 | 3 | CB-2 | 45 | C-1 | 50 | D-1/D-2 | 5,500/1,500 |
| Example 55 | R-55 | A-1 | 100 | F-1 | 3 | CB-3 | 45 | C-1 | 50 | D-1/D-2 | 5,500/1,500 |
| Example 56 | R-56 | A-1 | 100 | F-1 | 3 | B-12 | 45 | C-1 | 50 | D-1/D-2 | 5,500/1,500 |
| Example 57 | R-57 | A-1 | 100 | F-1 | 3 | B-13 | 45 | C-1 | 50 | D-1/D-2 | 5,500/1,500 |
| Example 58 | R-58 | A-1 | 100 | F-1 | 3 | B-1 | 45 | C-8 | 50 | D-1/D-2 | 5,500/1,500 |
| Example 59 | R-59 | A-1 | 100 | F-1 | 3 | B-1 | 45 | C-9 | 50 | D-1/D-2 | 5,500/1,500 |
| Comparative | CR-1 | A-1 | 100 | F-1 | 3 | CB-1 | 45 | CC-1 | 50 | D-1/D-2 | 5,500/1,500 |
| Example 1 | | | | | | | | | | | |
| Comparative | CR-2 | CA-1 | 100 | F-1 | 3 | B-1 | 45 | C-1 | 50 | D-1/D-2 | 5,500/1,500 |
| Example 2 | | | | | | | | | | | |
| Comparative | CR-3 | CA-1 | 100 | F-1 | 3 | CB-1 | 45 | CC-1 | 50 | D-1/D-2 | 5,500/1,500 |
| Example 3 | | | | | | | | | | | |

### Evaluations

Each radiation-sensitive resin composition prepared as described above was evaluated on the sensitivity, the CDU performance, and the resolution in accordance with the following methods.

### Sensitivity

An underlayer antireflective film having an average thickness of 105 nm was formed on a 12-inch silicon wafer by applying a composition for forming an underlayer antireflective film (“ARC66” available from Brewer Science) on the surface of the 12-inch silicon wafer using a spin-coater (“CLEAN TRACK ACT 12” available from Tokyo Electron Limited), and thereafter heating the composition at 205° C. for 60 sec. Each radiation-sensitive resin composition shown in Table 2 was applied on the underlayer antireflective film using the spin-coater, and subjected to PB at 130° C. for 60 sec. Thereafter, cooling was performed at 23° C. for 30 sec to form a resist film having an average thickness of 45 nm. This resist film was subjected to exposure by using an EUV scanner (“NXE3300” manufactured by ASML Co., (NA=0.33; σ=0.9/0.6; quadrupole illumination, with a hole pattern mask having a pitch of 50 nm, +20% bias in terms of the dimension on the wafer). PEB was performed for 60 sec on a 100° C. hot plate, and development was carried out for 30 seconds with a 2.38% by mass aqueous solution of tetramethylammonium hydroxide to form a resist pattern with 25 nm holes and 50 nm pitch (hereinafter, may be also referred to as “25 nm contact hole pattern”). An exposure dose at which the 25 nm contact hole pattern is formed was defined as an optimum exposure dose, and this optimum exposure dose was adopted as the sensitivity (mJ/cm²). The sensitivity having a lower value indicates more favorability. The sensitivity was evaluated to be: “A” (extremely favorable) in a case of less than 62 mJ/cm²; “B” (favorable) in a case of no less than 62 mJ/cm² and no greater than 65 mJ/cm²; and “C” (unfavorable) in a case of being greater than 65 mJ/cm².

### CDU Performance

An irradiation was performed at the optimum exposure dose determined in the above section “Sensitivity”, and a 25 nm contact hole pattern was formed similarly to that described above. The 25 nm contact hole pattern of the resist pattern formed was observed from above by using a scanning electron microscope (“CG-5000,” available from Hitachi High-Technologies Corporation), and hole diameters at a total of 800 arbitrary points were measured. Variance (3σ) of the dimensions was determined, and this was adopted as CDU (nm). The CDU value being smaller indicates more favorable CDU performance, revealing less variance of the hole diameters in greater ranges. The CDU performance was evaluated to be: “A” (extremely favorable) in a case of the CDU being less than 3.6 nm; “B” (favorable) in a case of the CDU being no less than 3.6 nm and less than 3.8 nm; and “C” (unfavorable) in a case of the CDU being no less than 3.8 nm.

### Resolution

A minimum diameter on the contact hole pattern resolved with varying exposure doses was measured in the method described in the above section “Sensitivity”, and the measurement value was adopted as resolution (unit: nm). The resolution value being smaller indicates more favorable resolution. The resolution was evaluated to be: “A” (extremely favorable) in a case of the resolution being less than 20.0 nm; “B” (favorable) in a case of the resolution being no less than 20.0 nm and no greater than 21.0 nm; and “C” (unfavorable) in a case of the resolution being greater than 21.0 nm.

TABLE 3

| | Radiation-sensitive | | CDU | |
|---|---|---|---|---|
| | resin composition | Sensitivity | performance | Resolution |
| Example 1 | R-1 | B | A | B |
| Example 2 | R-2 | B | A | B |
| Example 3 | R-3 | B | A | B |
| Example 4 | R-4 | B | B | A |
| Example 5 | R-5 | B | A | B |
| Example 6 | R-6 | B | A | A |
| Example 7 | R-7 | B | A | B |
| Example 8 | R-8 | A | A | B |
| Example 9 | R-9 | A | A | B |
| Example 10 | R-10 | B | B | B |
| Example 11 | R-11 | B | B | B |
| Example 12 | R-12 | B | A | B |
| Example 13 | R-13 | B | A | A |
| Example 14 | R-14 | A | A | A |
| Example 15 | R-15 | B | B | A |
| Example 16 | R-16 | A | A | B |
| Example 17 | R-17 | A | B | A |
| Example 18 | R-18 | B | A | B |
| Example 19 | R-19 | B | A | A |
| Example 20 | R-20 | B | B | B |
| Example 21 | R-21 | B | A | A |
| Example 22 | R-22 | B | A | A |
| Example 23 | R-23 | B | A | A |
| Example 24 | R-24 | B | A | A |
| Example 25 | R-25 | A | B | B |
| Example 26 | R-26 | B | A | B |
| Example 27 | R-27 | B | A | B |
| Example 28 | R-28 | B | A | A |
| Example 29 | R-29 | B | B | B |
| Example 30 | R-30 | B | A | B |
| Example 31 | R-31 | B | A | A |
| Example 32 | R-32 | B | A | A |
| Example 33 | R-33 | B | B | B |
| Example 34 | R-34 | A | A | A |
| Example 35 | R-35 | B | A | A |
| Example 36 | R-36 | A | A | A |
| Example 37 | R-37 | B | A | A |
| Example 38 | R-38 | B | A | B |
| Example 39 | R-39 | A | A | A |
| Example 40 | R-40 | A | A | A |
| Example 41 | R-41 | A | A | A |
| Example 42 | R-42 | B | A | B |
| Example 43 | R-43 | B | A | B |
| Example 44 | R-44 | A | A | A |
| Example 45 | R-45 | A | A | A |
| Example 46 | R-46 | B | B | B |
| Example 47 | R-47 | B | B | B |
| Example 48 | R-48 | B | B | B |
| Example 49 | R-49 | B | A | B |
| Example 50 | R-50 | B | A | B |
| Example 51 | R-51 | B | A | B |
| Example 52 | R-52 | B | A | A |
| Example 53 | R-53 | B | A | B |
| Example 54 | R-54 | B | B | B |
| Example 55 | R-55 | B | B | B |
| Example 56 | R-56 | B | A | B |
| Example 57 | R-57 | A | A | B |
| Example 58 | R-58 | B | A | B |
| Example 59 | R-59 | A | A | B |
| Comparative | CR-1 | C | C | C |
| Example 1 | | | | |
| Comparative | CR-2 | C | C | C |
| Example 2 | | | | |
| Comparative | CR-3 | C | C | C |
| Example 3 | | | | |

Obviously, numerous modifications and variations of the present invention(s) are possible in light of the above teachings. It is therefore to be understood that within the scope of the appended claims, the invention(s) may be practiced otherwise than as specifically described herein.

What is claimed is:

1. A radiation-sensitive resin composition comprising:
a first polymer comprising a first structural unit comprising a partial structure obtained by substituting a hydrogen atom of a carboxy group or of a phenolic hydroxy group with an acid-labile group represented by formula (1); and
a compound comprising: a monovalent radiation-sensitive onium cation moiety comprising an aromatic ring structure which comprises a fluorine atom or a fluorine atom-containing group; and a monovalent organic acid anion moiety, 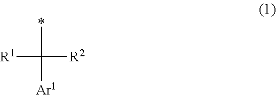
wherein, in the formula (1),
Ar¹ represents a group obtained by removing one hydrogen atom from a substituted or unsubstituted aromatic ring structure having 5 to 30 ring atoms;
R¹ and R² each independently represent a substituted or unsubstituted monovalent aliphatic hydrocarbon group having 1 to 10 carbon atoms; and
* denotes a site bonding to an ethereal oxygen atom in the carboxy group or an oxygen atom in the phenolic hydroxy group.

2. The radiation-sensitive resin composition according to claim 1, wherein the substituted or unsubstituted aromatic ring structure having 5 to 30 ring atoms which gives Ar¹ in the formula (1) is a substituted or unsubstituted aromatic hydrocarbon ring structure having 6 to 30 ring atoms.

3. The radiation-sensitive resin composition according to claim 1, wherein R¹ and R² in the formula (1) each independently represent a substituted or unsubstituted monovalent chain hydrocarbon group having 1 to 10 carbon atoms.

4. The radiation-sensitive resin composition according to claim 1, wherein the radiation-sensitive onium cation moiety is represented by formula (2-1) or (2-2): 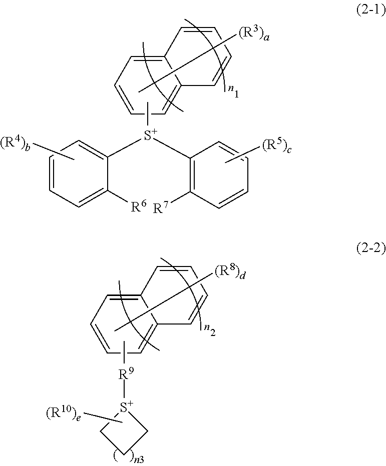
wherein,
in the formula (2-1),
a is an integer of 0 to 7, b is an integer of 0 to 4, and c is an integer of 0 to 4, wherein a sum of a, b, and c is no less than 1;
R³, R⁴, and R⁵ each independently represent a halogen atom, a hydroxy group, a nitro group, or a monovalent organic group having 1 to 20 carbon atoms, wherein at least one of R³, R⁴, and R⁵ represents a fluorine atom or a monovalent fluorinated hydrocarbon group having 1 to 10 carbon atoms;
in a case in which a is no less than 2, a plurality of R³s are identical or different from each other, in a case in which b is no less than 2, a plurality of R⁴s are identical or different from each other, and in a case in which c is no less than 2, a plurality of R⁵s are identical or different from each other;
R⁶ and R⁷ each independently represent a hydrogen atom, a fluorine atom, or a monovalent fluorinated hydrocarbon group having 1 to 10 carbon atoms, or R⁶ and R⁷ taken together represent a single bond; and
n₁ is 0 or 1, and
in the formula (2-2),
d is an integer of 1 to 7 and e is an integer of 0 to 10, wherein in a case in which d is 1, R⁸ represents a fluorine atom or a monovalent fluorinated hydrocarbon group having 1 to 10 carbon atoms, and in a case in which d is no less than 2, a plurality of R⁸s are identical or different from each other, and each R⁸ represents a halogen atom, a hydroxy group, a nitro group, or a monovalent organic group having 1 to 20 carbon atoms, wherein at least one of the plurality of R⁸s represents a fluorine atom or a monovalent fluorinated hydrocarbon group having 1 to 10 carbon atoms;
R⁹ represents a single bond or a divalent organic group having 1 to 20 carbon atoms;
R¹⁰ represents a halogen atom, a hydroxy group, a nitro group, or a monovalent organic group having 1 to 20 carbon atoms, wherein in a case in which e is no less than 2, a plurality of R¹⁰s are identical or different from each other;
n₂ is 0 or 1; and
n₃ is an integer of 0 to 3.

5. The radiation-sensitive resin composition according to claim 1, wherein the first structural unit is represented by formula (3-1) or (3-2): 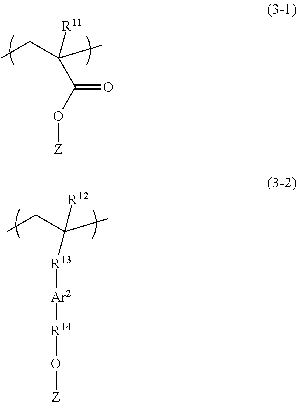
wherein,
in the formulae (3-1) and (3-2), Z represents the acid-labile group represented by the formula (1);
in the formula (3-1), R¹¹ represents a hydrogen atom, a fluorine atom, a methyl group, or a trifluoromethyl group; and
in the formula (3-2),
R¹² represents a hydrogen atom or a methyl group;
R¹³ represents a single bond, an oxygen atom, —COO—, or —CONH—;
Ar² represents a group obtained by removing two hydrogen atoms from a substituted or unsubstituted aromatic hydrocarbon ring structure having 6 to 30 ring atoms; and
R¹⁴ represents a single bond or —CO—.

6. The radiation-sensitive resin composition according to claim 1, wherein the first polymer further comprises a second structural unit comprising a phenolic hydroxy group.

7. The radiation-sensitive resin composition according to claim 1, further comprising a second polymer having a percentage content of fluorine atoms which is greater than a percentage content of fluorine atoms of the first polymer.

8. A method of forming a resist pattern, the method comprising:
applying the radiation-sensitive resin composition according to claim 1 directly or indirectly on a substrate to form a resist film;
exposing the resist film; and
developing the resist film exposed.
